(19) United States
(12) Patent Application Publication
(10) Pub. No.: US 2026/0265810 A1
(43) Pub. Date: Sep. 10, 2026

(54) TAGGING NUCLEIC ACIDS FOR SEQUENCE ASSEMBLY

(71) Applicant: Dovetail Genomics, LLC, Scotts Valley, CA (US)

(72) Inventor: Richard E. GREEN, Santa Cruz, CA (US)

(21) Appl. No.: 19/660,829

(22) Filed: Apr. 28, 2026

Related U.S. Application Data
(63) Continuation-in-part of application No. 16/605,158, filed on Apr. 17, 2018, now abandoned.
(60) Provisional application No. 62/486,803, filed on Apr. 18, 2017.

(51) Int. Cl. C12Q 1/6806; C12Q 1/6874

(52) U.S. Cl. CPC C12Q 1/6806; C12Q 1/6874

(57) ABSTRACT

Various approaches for generating long-distance contiguity information to facilitate contig assembly and phase determination are disclosed. Nucleic acids are assembled into complexes using binding moieties such that, when the nucleic acid backbones are cleaved, the ensuing fragments remain bound. Exposed ends are tagged and ligated either to one another or to tagging moieties such as oligo labels. Ligated junctions are sequenced, and the sequence information is used to assemble contigs into common scaffolds or to assign phase information. Various approaches to tagging the exposed ends are presented.

### CROSS REFERENCE

This application is a continuation-in-part of U.S. patent application Ser. No. 18/949,657, filed Nov. 15, 2024, which is a continuation of U.S. patent application Ser. No. 16/685,855, filed Nov. 15, 2019, now U.S. Pat. No. 12,180,535, which is a continuation of U.S. patent application Ser. No. 15/329,414, filed Jan. 26, 2017, now U.S. Pat. No. 10,526,641, which is a national stage entry of International Application No. PCT/US2015/043327, filed Jul. 31, 2015, which claims the benefit under 35 U.S.C. § 119 of U.S. Provisional Application Ser. No. 62/032,139, filed Aug. 1, 2014, the contents of which are hereby incorporated by reference in their entirety, U.S. Provisional Application Ser. No. 62/032,166, filed Aug. 1, 2014, the contents of which are hereby incorporated by reference in their entirety, U.S. Provisional Application Ser. No. 62/032,181, filed Aug. 1, 2014, the contents of which are hereby incorporated by reference in their entirety, and U.S. Provisional Application Ser. No. 62/032,221, filed Aug. 1, 2014, the contents of which are hereby incorporated by reference in their entirety.

### BACKGROUND OF THE INVENTION

High-throughput sequencing allows genetic analysis of the organisms that inhabit a wide variety of environments of biomedical, ecological, or biochemical interest. Shotgun sequencing of environmental samples, which often contain microbes that are refractory to culture, can reveal the genes and biochemical pathways present within the organisms in a given environment. Careful filtering and analysis of these data can also reveal signals of phylogenetic relatedness between reads in the data. However, high-quality de novo assembly of these highly complex datasets is generally considered to be intractable.

### SUMMARY OF THE INVENTION

Metagenomics is the study of the genomes present in living communities that may contain many tens, hundreds, or thousands of individual species. Each of these species may be present in vastly different numbers. Thus, DNA collected from metagenomic samples presents unique challenges for de novo assembly. Combining proximity-ligation data (Chicago data) with shotgun sequencing data can improve the contiguity of metagenomic assemblies, enabling greater biological understanding of the ecology, evolution, and biochemical potential in these communities, as is described in the following patent references. US Patent No. U.S. Pat. No. 9,411,930 filed Jan. 31, 2014, issued Aug. 9, 2016 is hereby incorporated herein in its entirety. US Patent Application Publication No. US20150363550, published Dec. 17, 2015 is hereby incorporated by reference in its entirety. PCT Application No. PCT/US2014/014184 filed Jan. 31, 2014, published as WO 2014/121091 on Aug. 7, 2014, is hereby incorporated by reference in its entirety. PCT Application No. PCT/US2016/057557 filed Oct. 18, 2016, published as WO 2017/070123 on Apr. 27, 2017, is hereby incorporated herein in its entirety. Described herein are additional methods and enhancements of such metagenomic assembly methods that exploit the varied patterns of base modifications present within microbial and other genomes. For example, some methods use a combination of restriction enzymes that have different sensitivities to specific base modifications, such as methylation, to generate Chicago or other libraries. The resulting sequence data can reveal which genomic segments can and cannot be derived from the same strain or species. Incorporating these data into a computational genome assembly strategy allows for more complete genome assemblies and allow partitioning of these assemblies according to which base modifications are present.

A feature of microbial and eukaryotic genomes is their use of base-modifications to regulate gene expression (eukaryotes) or to mark and protect their genomes from endogenous restriction enzymes that they use for clearing foreign DNA (prokaryotes). These base modifications can include CpG methylation of cytosines, methylation of adenosine (dam methylation) or methylation of cytosine (dcm methylation) in specific, small sites. When these modifications are present, they can prevent the action of some restriction enzymes. In this way, some microbes protect their genomes from their own defensive enzymes that they can then use to degrade any invading DNA.

Methods and compositions disclosed herein exploit the differential base modifications present in the various genomes in metagenomic communities to improve genome assembly and determine which assembled sequences derive from strains or species that have these base modification systems.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings (Figures or FIG.) of which:

FIG. 1A shows a metagenomic assembly that is made using a cocktail of three isoschizomer restriction enzymes: MboI, DpnI, and Sau3 AI.

FIG. 1B shows a metagenomic assembly that is made using only MboI, which is sensitive to dam methylation.

FIG. 2A shows an exemplary schematic of a procedure for proximity ligation.

FIG. 2B shows an exemplary schematic of two pipelines for sample preparation for metagenomic analysis.

### DETAILED DESCRIPTION

Disclosed herein are methods and compositions for the assembly of nucleic acid data into scaffolds. The disclosure herein supplements assembly approaches by providing epigenomic, other non-sequence and non-alignment-based methods or supplements to methods of sequence and contig assembly. Practice of methods disclosed herein facilitates more accurate assignment of single read or multi-read contig information into scaffolds or into higher-order genomic groupings, even in the absence of overlapping sequence or paired-end reads.

Through practice of the methods and use of compositions disclosed herein, nucleic acid sequence is sorted such that sequences, such as contigs or scaffolds, arising from a common source such as a common genome in a heterogeneous sample comprising multiple genomic nucleic acid sources, or a common chromosome in a sample comprising a plurality of chromosomes or chromosome types, are accurately and rapidly assigned to a genomic source or a common scaffold. Assignment is in some cases informed by a genome characteristic, for example DNA modification such as methylation, or by a skewed or distinctive GC frequency, or by the impact of such characteristic on library generation using sample digestion relying upon a restriction endonuclease that is sensitive to such characteristic.

Nucleic acid samples for which methods and compositions here facilitate assembly include heterogeneous samples such as environmental samples, gut samples, blood samples such as those obtained from an individual or individuals suspected of sharing a common disorder or communicable disease. Alternately, samples from a relatively homogeneous source such as a single individual are beneficially assembled herein through the identification and employment of chromosome or sub-chromosomal features such as inter-chromosomal or intra-chromosomal variation in repeat frequency, transposon content, methylation frequency or other chromosomal-specific feature.

In various embodiments herein, a factor common to a subset of nucleic acid molecules in a sample, such as molecules arising from a common chromosome or from a common genome, is identified, and sequences such as single reads, contigs or scaffolds are grouped according to the presence or relative abundance of an identified feature.

Some features contemplated herein are identified through examination or analysis of sequence information. Exemplary features include GC content (or, complementarily, AT content), repeat sequence or frequency, such as k-mer repeat, Alu, microsatellite, transposon or other repeat, or codon selection bias for identified coding regions or mRNA or cDNA transcripts.

Alternately or in combination, epigenetic features such as sequence specific methylation patters or aggregate methylation frequency are used to inform sequence, contig or scaffold assembly. In these cases, assembly is improved in through identification of a subset of molecules having a common modification, such as an increased methylation frequency, and grouping sequence from these molecules into a common putative genome or chromosome of origin.

Often, the feature is common to an organism, such as an organism having a distinctive GC content, repeat content or methylation frequency. Plasmodium species, for example, have a distinctive GC contend of often less than 30%, facilitating identification of sequences from this source in a heterogeneous sample. Similarly, dinoflagellate genomes are regularly highly methylated, a fact which has complicated efforts at sequencing. Features are observed having a frequency of no more than 10%, no more than 20%, no more than 30%, no more than 50%, no more than 70%, or at most 10%, at most 20%, at most 30%, at most 50%, or at most 70% or greater.

Alternately, in some cases a single chromosome of an organism is differentially characterized relative to other chromosomes of that organism. For example Y-chromosomes are often repeat rich, while X-chromosomes in females are often differentially methylated or otherwise silenced. Alternately, in some species chromosomes exhibit differential GC content, such as the putative sex chromosome of the unicellular alga Ostreococcus.

In exemplary embodiments, the feature is an epigenetic modification. Exemplary epigenetic modifications include methylation, such as CpG methylation in eukaryotes such as mammals, dam and dem methylation in some eubacteria, and a range of additional methylation and other epigenomic modifications.

When the feature is identified through scrutiny of the sequence, such as GC content or repeat frequency, it is readily identified through sequence analysis such as direct sequencing or sequencing supported by analysis such as machine learning or other pattern recognition approaches.

Alternately, in cases where a feature is not readily ascertained through direct sequencing, such as epigenetic modifications, direct molecular biology approaches are used to identify or characterize the abundance or distribution of a feature. In such cases, a feature such as methylation frequency is ascertained, for example, by differential digestion using restriction endonucleases. Optionally, isoschizomers that cut a common target sequence but exhibit differential sensitivity to methylation within the cut site are used to assemble sequencing libraries. A sample is optionally aliquoted and differentially subjected to digestion using isoschizomers differing in methylation sensitivity, and the results are analyzed for an impact on the resulting library. In some cases the library is a ‘Hi-C’ or ‘Chicago’ library generation protocol as taught in U.S. Pat. No. 9,411,930, issued Apr. 21, 2015, which is hereby incorporated by reference in its entirety, modified herein so as to effect the methods disclosed herein.

Under some such examples, digestion is effected using isoschizomers MboI, DpnI and Sau3 A1. All enzymes cut a common sequence, but MboI alone among the set is sensitive to dam methylation. By subjecting the sample to digests comprising, for example, all three enzymes or DpnI or Sau3A1 alone, in comparison with a digest using MboI alone the isoschizomer list (optionally supplemented din both cases with additional restriction endonuclease activity that is not isoschizomeric to the set used herein), one may visualize an impact of methylation on the MboI library relative to the non-sensitive library. That is, by identifying library components that differ in their border sequences between MboI and aggregate digestions, one identifies sequences arising from molecules subject to differential methylation. Contigs to which said sequences map are optionally separated from contigs having sequence that is not differentially methylated, and assigned to a common chromosome or genome, or is otherwise separated from the unmethylated contig set. Alternately, if methylation is observed to be relatively frequent in the set, contigs corresponding to unmethylated nucleic acid sources are grouped and assigned a common source.

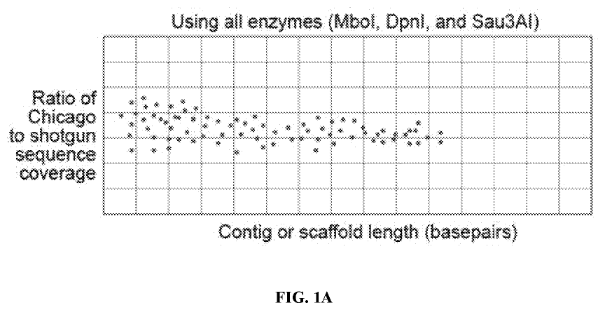

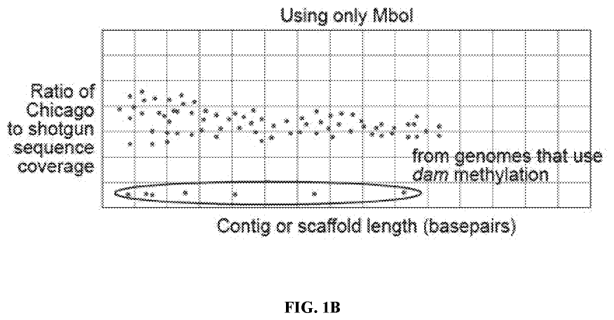

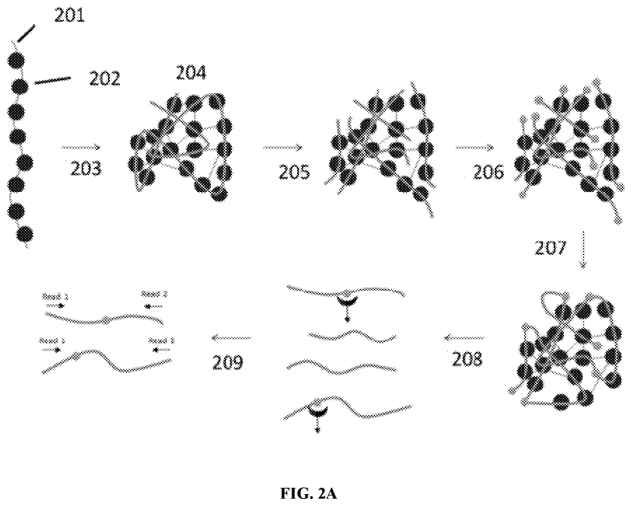

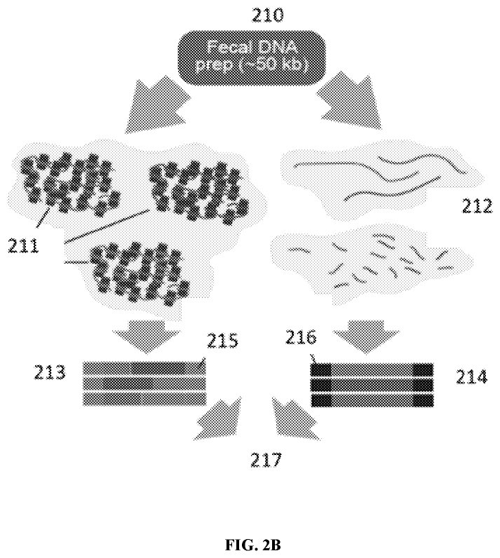

Often contigs are clustered according to their nucleic acid composition or modification state, such as methylation state, based on the corresponding sequencing reads being present in Chicago libraries generated by the specified restriction enzyme (as exemplified in FIG. 1A and FIG. 1B). FIG. 1A and FIG. 1B depict a method for identifying assembled sequences that derive from strains or species that are dam methylated. FIG. 1A shows a metagenomic assembly, as generated using the protocol in FIG. 2B, and was made using a cocktail of all isoschizomer restriction enzymes listed in Table 2. The ratio of Chicago/shotgun reads, per contig (y-axis) is nearly constant across contigs because all instances of GATC are cut with at least one of the restriction enzymes. FIG. 1B shows that when the Chicago library is generated using an enzyme, MboI for example, that is sensitive to dam methylation, the ratio of Chicago to shotgun reads is severely reduced in genomes that are dam methylated. In this way, those components are identified as belonging to strains or species that use dam methylation. In light of the above, more generally, disclosed herein are approaches for contig assembly that are informed by nucleic acid composition or modification state such as methylation state. Libraries are generated using approaches that are independent of DNA modification status, and using approaches that are impacted by modification status. The number or normalized number of reads, or representation of a given read set in the population (such as reads adjacent to potential modification-sensitive cleavage sites) is compared to a similar metric obtained from a library generated using a modification sensitive approach, such as a digestion regimen involving an enzyme of Table 1. Read pairs or other read sequence information that is unaffected by the use of a modification sensitive enzyme is inferred to map to contigs that represent nucleic acid molecules not modified at that site. Alternately, reads or read pairs that demonstrate a differential abundance (such as a lower abundance, relative abundance or other measure of frequency or normalized frequency) indicate that the contigs to which they map are likely to be differentially modified at the enzyme recognition sites. Using this approach, contigs of unknown origin are assigned to an organism having a modification or GC abundance status comparable to that of the contigs at the site. Alternately, when organism identity or modification status is unknown, contigs that may or may not otherwise assemble into a common scaffold are nonetheless assigned to a common scaffold, genome or organism of origin, according to whether the contigs exhibit a shared modification such as methylation patters or frequency, relative to other contigs of a heterogeneous sample. See again FIG. 1A and FIG. 1B.

Grouping in some cases indicates a common genome or a common nucleic acid of origin, but in some cases a sample such as a heterogeneous sample may have more than one differentially methylated genome, such that grouping does not necessarily imply a common genomic or chromosomal source. Nevertheless, even in these cases, sorting based upon methylation, repeat frequency, GC content or other feature as disclosed herein or otherwise known or identified in the art, in some cases greatly facilitates contig, scaffold or genome assembly. In these cases, feature-sorting still simplifies assembly as it reduces the overall complexity of the contigs or scaffolds to be assessed for inclusion on one or another putative genome in a sample.

Alternately, some embodiments of the disclosure herein utilize an informatics approach to using nucleic acid characteristics modifications to facilitate or improve sequence or contig assembly into scaffolds or into larger groupings such as genome equivalent groupings. Nucleic acid information such as sequence information generated from bulk sequencing, shotgun sequencing or other sequencing of a heterogeneous sample is generated or obtained from a sequencing effort. In some cases the sequence information is generated through an approach that comprises use of a reagent such as a restriction endonuclease, nickase, transposase, phosphodiester backbone cleaving enzyme or repair enzyme that leads to, modulates or regulates nucleic acid cleavage, wherein the reagent has or regulates an activity that is not sensitive to a DNA modifying activity.

Sequence information is scrutinized so as to identify an open reading frame, coding region, coding region partial segment or other information indicative of a DNA modifying activity encoded in the sequence. Exemplary enzymes to be detected include but are not limited to enzymes having a capacity to transfer a methyl group to (‘to methylate’) CpG islands, dam methylation sites or dem methylation sites, or to acetylate, alkylate, phosphorylate or otherwise to modify DNA.

A reagent is selected, such as a restriction endonuclease, nickase, transposase, phosphodiester backbone cleaving enzyme or repair enzyme, that leads to, modulates or regulates nucleic acid cleavage, and having or regulating an activity that is sensitive to a DNA feature such as GC abundance or a DNA modifying activity encoded in the sequence. Following the examples above, such an enzyme is in some cases an enzyme having an activity that is sensitive to or impacted by methylation at CpG islands, dam methylation or dem methylation, or to acetylation, alkylation, phosphorylation or other DNA modification. The reagent is often isoschizomeric to a reagent selected in the initial library preparation or sequencing effort, but differentially affected by presence of the DNA modification.

The differentially affected reagent is used in a sequencing or library generation. Often, the library preparation is performed under the same or comparable conditions, differing only in the use of the modification-sensitive isoschizomer reagent. Alternately, additional changes are introduced in the sequencing or library preparation without substantially impacting the fact that the first and second sequencing or library preparation differ in the presence of a modification sensitive reagent.

Sequencing results for the second sequencing effort are generated or obtained. Comparison of the sequence data in the presence and absence of the sensitive reagent are compared. Often, the reagent is a methylation sensitive restriction endonuclease, such as MboI in place of Sau3A1. Sequence reads, contigs or scaffolds are identified that exhibit a difference in nucleic acid cleavage that correlates with a modification of the type found or hypothesized to be encoded by at least one locus in the sample. In some cases the differences are confirmed to correlate to positions likely to be impacted by the DNA modifying activity identified in the sequence.

Sequence reads, contigs, scaffolds or other nucleic acid sequence groupings are sorted as to whether a sequence read, contig, scaffold or other sequence grouping is differentially impacted by the presence and absence of the sensitive reagent such as a methylation sensitive restriction endonuclease. Sequence reads, contigs, scaffolds and other sequence groupings identified as being differentially impacted are grouped separately from sequence reads, contigs, scaffolds and other sequence groupings that are not differentially impacted, so as to inform sequence assembly of sequences generated from the heterogeneous sample.

In particular, sequence data sharing the modification impact are often assigned to a common genome, or are assigned to at least one genome distinct from sequence that does not exhibit the effect. Alternately or in combination, particularly when the effect is hypothesized to be relatively infrequent in a genome, sequence data exhibiting the effect are assigned to a common genome or at least one common genome. Sequence from which the modifying activity was identified, such as the open reading frame, coding sequence, coding sequence fragment or other sequence indicative of the activity is optionally also included in the grouping such as the putative genome grouping with the sequence exhibiting the differential effect, as is sequence that scaffolds with the sequence from which the modifying activity was identified.

Sequences exhibiting the differential effect will often vary according to the degree to which the effect is exhibited. That is, in some cases one observes sequences that are not differentially effected, sequences that are differentially effected at a first frequency or frequency range, and sequences that are effected at a second frequency or frequency range. In these cases, sequence data is stratified not only as to presence/absence of the sequence effect, but as to extend of effect, such as percent of putative modification sites affected. In these cases, sequences are sorted and assembled into putative genomes, chromosomes or chromosome regions based upon both presence and frequency of modification occurrence. That is, a sequence data set having unaffected contigs, contigs affected at 10% of potential dam sites and contigs affected at 70% of potential dam sites is sorted into three groupings, corresponding to at least three genomes of the original heterogeneous source. Alternately, the sequences are sorted into at least three chromosomes according to methylation frequency, or the sequences are sorted such that unmodified contigs are assigned to euchromatic regions, moderately modified contigs are assigned to heterochromatin, and highly modified contigs are assigned to, for example, centromeric or telomeric positions.

Thus, through practice of the methods herein, genome or other nucleic acid library assembly is simplified, allowing more accurate assembly, in less time, using less computational capacity.

### Further Discussion of Approaches and Applications of the Present Disclosure

Microbial contents of biological or biomedical samples, ecological or environmental samples, and food samples are frequently either identified or quantified through culture dependent methods. A significant amount of microbial biodiversity can be overlooked by cultivation-based methods as many microbes are unculturable, or not amenable to culture in the lab. Shotgun metagenomic sequencing approaches, in which thousands of organisms are sequenced in parallel, can allow researchers to comprehensively sample a majority of genes in a majority of organisms present in a given complex sample. This approach can enable the evaluation of bacterial diversity and the study of unculturable microorganisms that can otherwise be difficult to analyze. However, unsupported shotgun sequencing methods generate a significant number of reads comprising short read sequences that can be difficult to assemble without a reference sequence or without some source of long-range linkage information as needed to assemble sequences de novo.

Microbial communities are often comprised of tens, hundreds, or thousands of recognizable operational taxonomic units (OTUs), at very uneven abundance, each with varying amounts of strain variation. Further compounding the problem, microbes frequently exchange genetic materials through various means of conjugal exchange, and these segments of genetic material can be incorporated into the chromosomes of their hosts, resulting in rampant horizontal gene transfer within bacterial communities. Thus, microbial genomes are often described in terms of a core genome of genes that are widely present and others that may or may not be present in a particular strain. Describing the constituent genomes from and dynamics of a complex microbial community, such as the human gut microbiome, is an important and difficult challenge.

As a result of the difficulty of de novo metagenomic assembly, several simpler approaches have been developed and widely adopted to interrogate and describe their components. For example, 16S RNA amplification and sequencing is a common way to assess the community composition. While this approach can be used in a comparative framework to describe the dynamics of microbial communities before and after various stimuli or treatments, it provides a very narrow view of actual community composition since nothing is learned about the actual genomes outside their 16S regions. Binning approaches have also proved useful for classifying shotgun reads or contigs assembled from them. These approaches are useful for getting a provisional assignment of isolated genomic fragments to OTUs. However, they are essentially hypothesis generators and are powerless to order and orient these fragments or to assign fragments to strains within an OTU. Importantly, they are ill-suited to identify horizontally transferred sequences, since they detect OTU-of-origin rather than current linkages. From this perspective, these binning approaches based on k-mer occurrence, sequencing depth, and other features are a stop-gap method to understand isolated metagenomics components because highly contiguous assembly has heretofore not been possible in a reliable, fast, and economically reasonable way.

Disclosed herein are methods and tools for genetic analysis of organisms in metagenomic samples, such as microbes that cannot be cultured in a laboratory environment and that inhabit a wide variety of environments. The present disclosure provides methods of de novo genome assembly of read data from complex metagenomics datasets comprising connectivity data. Methods and compositions disclosed herein generate scaffolding data that uniformly and completely represents the composite species in a metagenomics sample.

FIG. 2A shows a schematic of a procedure for proximity ligation. DNA 201, such as high molecular weight DNA, is incubated with histones 202, and then crosslinked 203 (e.g., with formaldehyde) to form a chromatin aggregate 204. This locks the DNA molecules into a scaffold for further manipulation and analysis. The DNA is then digested 205, and digested ends are filled in 206 with a marker such as biotin. Marked ends are then randomly ligated to each other 207, and the ligated aggregate is then liberated 208, for example by protein digestion. The markers can then be used to select for DNA molecules containing ligation junctions 209, such as through streptavidin-biotin binding. These molecules can then be sequenced, and the reads in each read pair derive from two different regions of the source molecule, separated by some insert distance up to the size of the input DNA.

FIG. 2B shows two pipelines for sample preparation for metagenomic analysis, which can be employed separately or together. A single DNA preparation 210 (e.g., from fecal samples) is input into the process. In the case of fecal samples, collected DNA can be in approximately 50 kilobase fragments, such as from a preparation using the Qiagen fecal DNA kit. From this DNA, in vitro chromatin assemblies 211 (e.g., “Chicago”) and shotgun 212 libraries preparations can be made. In an exemplary embodiment, the present disclosure provides an approach that uses a combination of restriction enzymes that has different sensitivities to specific base modifications to generate Chicago libraries. For example, certain restriction enzymes that have different sensitivities to methylation, such as CpG methylation of cytosines, methylation of adensine (dam methylation) and methylation of cytosine (dcm methylation), can be used to generate Chicago libraries, improve genome assembly and determine which assembled sequences derive from strains or species that have particular base modification systems. The chromatin assembly library 213 and the shotgun library 214 can use different barcodes215 and 216 from each other. These two libraries can then be pooled for sequencing 217. Using such a protocol, a single DNA prep can serve as input for two sequencing libraries: shotgun and in vitro chromatin assembly. Less than 1 μg of input DNA is required to generate both libraries, and these libraries can be individually barcoded for pooling during sequencing. These data can then be assembled first into contigs and then scaffolded using the long-range linkage information from the in vitro chromatin assembly libraries. These data alone can generate many scaffolds of greater than one megabase, enabling a much more comprehensive view of microbial genome structure and dynamics than is currently achievable. Processing time to go from sample to highly contiguous assemblies can be under one week.

Some embodiments of the subject methods comprise proximity ligation and sequencing of in vitro assembled chromatin aggregates comprising metagenomic DNA samples, or DNA samples from uncultured microorganisms obtained directly from a sample, such as, for example, a biomedical or biological sample, an ecological or environmental sample, a complex biological environment, or a food sample. In compatible embodiments, nucleic acids are assembled into complexes, bound, cleaved to expose internal double-strand breaks, labeled to facilitate isolation of break junctions, and re-ligated so as to generate paired end sequences that are sequenced. In some such paired end sequences, both ends of the paired end read are inferred to map to a common nucleic acid molecule, even if the sequences of the paired read map to distinct contigs.

In similarly preferred embodiments, exposed ends of bound complexes are tagged using identifiers such as nucleic acid barcodes, such that a complex is tagged or barcoded such that tag-adjacent sequence is inferred to likely arise from a single nucleic acid. Again, commonly barcoded sequences may map to multiple contigs, but the contigs are then inferred to map to a common nucleic acid molecule.

In similarly preferred embodiments, complexes are assembled through the addition of nucleic acid binding proteins other than histones, such as nuclear proteins, transposases, transcription factors, topoisomerases, specific or nonspecific double-stranded DNA binding proteins, or other suitable proteins. Alternately or in combination, complexes are assembled using nanoparticles rather than histones or other nucleic acid binding proteins.

In similarly preferred embodiments, natively occurring complexes are relied upon to preserve linkage information for nucleic acid complexes. In some such cases, nucleic acids are isolated so as to preserve complexes natively assembled, or are treated with a stabilizing agent such as a fixative prior to treatment or isolation.

In any assembled or isolated complex, cross-linking can be relied upon in some cases to stabilize nucleic acid complex formation, while in alternate cases the nucleic acid-binding moiety interactions are sufficient to maintain complex integrity in the absence of cross-linking.

The methods and compositions herein, alone or in combination with independently obtained or generated sequence data such as shotgun sequencing data, cab generate assemblies of genomic information for genomes, chromosomes or independent nucleic acid molecules in heterogeneous nucleic acid samples. Genomes can be assembled representing organisms, culturable or unculturable, such as abundant or rare organisms in a wide range of metagenomics communities, such as the human oral or gut microbiomes, and including organisms that are not amenable to growth in culture. Organisms can also be individuals in a sample with genetic material from a mixed group or population of other individuals, such as a sample containing cells or nucleic acids from multiple different human individuals. Methods of the present disclosure offer fast and simple approaches to high-throughput, culture-free assembly of genomes, in some cases using widely available high-throughput sequencing technology.

As used herein and in the appended claims, the singular forms “a,” “and,” and “the” include plural referents unless the context clearly dictates otherwise. Thus, for example, reference to “contig” includes a plurality of such contigs.

As used herein, “obtaining” a nucleic acid sample is given a broad meaning in some cases, such that it refers to receiving an isolated nucleic acid sample, as well as receiving a raw human or environmental sample, for example, and isolating nucleic acids therefrom.

The use of “and” means “and/or” unless stated otherwise. Similarly, “comprise,” “comprises,” “comprising,” “include,” “includes,” and “including” are interchangeable and not intended to be limiting, and refer to the nonexclusive presence of the recited element, leaving open the possibility that additional elements are present.

The term “read,” “sequence read,” or “sequencing read” as used herein, refers to the sequence of a fragment or segment of DNA or RNA nucleic acid that is determined in a single reaction or run of a sequencing reaction.

The term “contig” and “contigs” as used herein, refers to contiguous regions of DNA sequence assembled through common overlapping information. “Contigs” can be determined by any number methods known in the art, such as, by comparing sequencing reads for overlapping sequences, and/or by comparing sequencing reads against a databases of known sequences in order to identify which sequencing reads have a high probability of being contiguous.

The terms “polynucleotide,” “nucleotide,” “nucleic acid” and “oligonucleotide” are often used interchangeably. They generally refer to a polymeric form of nucleotides of any length, either deoxyribonucleotides or ribonucleotides, or analogs thereof. Polynucleotides comprise base monomers that are joined at their ribose backbones by phosphodiester bonds. Polynucleotides may have any three dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. Generally, an oligonucleotide comprises only a few bases, while a polynucleotide can comprise any number but is generally longer, while a nucleic acid can refer to a polymer of any length, up to and including the length of a chromosome or an entire genome. Also, the term nucleic acid is often used collectively, such that a nucleic acid sample does not necessarily refer to a single nucleic acid molecule; rather it may refer to a sample comprising a plurality of nucleic acid molecules. The term nucleic acid can encompass double- or triple-stranded nucleic acids, as well as single-stranded molecules. In double- or triple-stranded nucleic acids, the nucleic acid strands need not be coextensive, e.g., a double-stranded nucleic acid need not be double-stranded along the entire length of both strands. The term nucleic acid can encompass any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

The term “naked DNA” as used herein can refer to DNA that is substantially free of complexed DNA binding proteins. For example, it can refer to DNA complexed with less than about 10%, about 5%, or about 1% of the endogenous proteins found in the cell nucleus, or less than about 10%, about 5%, or about 1% of the endogenous DNA-binding proteins regularly bound to the nucleic acid in vivo, or less than about 10%, about 5%, or about 1% of an exogenously added nucleic acid binding protein or other nucleic acid binding moiety, such as a nanoparticle. In some cases, naked DNA refers to DNA that is not complexed to DNA binding proteins.

The terms “polypeptide” and “protein” are often used interchangeably and generally refer to a polymeric form of amino acids, or analogs thereof bound by polypeptide bonds. Polypeptides and proteins can be polymers of any length. Polypeptides and proteins can have any three dimensional structure, and may perform any function, known or unknown. Polypeptides and proteins can comprise modifications, including phosphorylation, lipidation, prenylation, sulfation, hydroxylation, acetylation, formation of disulfide bonds, and the like. In some cases “protein” refers to a polypeptide having a known function or known to occur naturally in a biological system, but this distinction is not always adhered to in the art.

As used herein, nucleic acids are “stabilized” if they are bound by a binding moiety or binding moieties such that separate segments of a nucleic acid are held in a single complex independent of their common phosphodiester backbone. Stabilized nucleic acids in complexes remain bound independent of their phosphodiester backbones, such that treatment with a restriction endonuclease does not result in disintegration of the complex, and internal double-stranded DNA breaks are accessible without the complex losing its integrity.

Alternately or in combination, nucleic acid complexes comprising nucleic acids and nucleic acid binding moieties are “stabilized” by treatment that increases their binding or renders them otherwise resistant to degradation or dissolution. An example of stabilizing a complex comprises treating the complex with a fixative such as formaldehyde or psorlen, or treating with UV light o as to induce cross-linking between nucleic acids and binding moieties, or among binding moieties, such that the complex or complexes are resistant to degradation or dissolution, for example following restriction endonuclease treatment or treatment to induce nucleic acid shearing.

The term “scaffold” as used herein generally refers to contigs separated by gaps of known length but unknown sequence or separated by unknown length but known to reside on a single molecule, or ordered and oriented sets of contigs that are linked to one another by mate pairs of sequencing reads. In cases where contigs are separated by gaps of known length, the sequence of the gaps may be determined by various methods, including PCR amplification followed by sequencing (for smaller gaps) and bacterial artificial chromosome (BAC) cloning methods followed by sequencing (for larger gaps).

The term “stabilized sample” as used herein refers to a nucleic acid that is stabilized in relation to an association molecule via intermolecular interactions such that the nucleic acid and association molecule are bound in a manner that is resistant to molecular manipulations such as restriction endonuclease treatment, DNA shearing, labeling of nucleic acid breaks, or ligation. Nucleic acids known in the art include but are not limited to DNA and RNA, and derivatives thereof. The intermolecular interactions can be covalent or non-covalent. Exemplary methods of covalent binding include but are not limited to crosslinking techniques, coupling reactions, or other methods that are known to one of ordinary skill in the art. Exemplary methods of noncovalent interactions involve binding via ionic interactions, hydrogen bonding, halogen bonding, Van der Waals forces (e.g. dipole interactions), π-effects (e.g. π-π interactions, cation-T and anion-π interactions, polar π interactions, etc.), hydrophobic effects, and other noncovalent interactions that are known to one of ordinary skill in the art. Examples of association molecules include, but are not limited to, chromosomal proteins (e.g. histones), transposases, and any nanoparticle that is known to covalently or non-covalently interact with nucleic acids.

The term “heterogeneous sample” as used herein refers a biological sample comprising a diverse population of nucleic acids (e.g. DNA, RNA), cells, organisms, or other biological molecules. In many cases the nucleic acids originate from one than one organism. For example, a heterogeneous nucleic acid sample can comprise at least about 1000, 2000, 3000, 4000, 5000, 6000, 7000, 8000, 9000, 10,000, 20,000, 50,000, 100,000, 200,000, 500,000, 1,000,000, 2,000,000, 5,000,000, 10,000,000, or more DNA molecules. Further, each of the DNA molecules can comprise the full or partial genome of at least one or at least two or more than two organisms, such that the heterogeneous nucleic sample can comprise the full or partial genome of at least about 1000, 2000, 3000, 4000, 5000, 6000, 7000, 8000, 9000, 10,000, 20,000, 50,000, 100,000, 200,000, 500,000, 1,000,000, 2,000,000, 5,000,000, 10,000,000, or more different organisms. Examples of heterogeneous samples are those obtained from a variety of sources, including but not limited to a subject's blood, sweat, urine, stool, or skin; or an environmental source (e.g. soil, seawater); a food source; a waste site such as a garbage dump, sewer or public toilet; or a trash can.

A “partial genome” of an organism can comprise at least about 10%, 20%, 30%, 40%, 50%, 60%, 70%, 80%, 90%, 95%, 99% or more the entire genome of an organism, or can comprise a sequence data set comprising at least about 10%, 20%, 30%, 40%, 50%, 60%, 70%, 80%, 90%, 95%, 99% or more of the sequence information of the entire genome.

The term “about” as used herein to describe a number, unless otherwise specified, refers to a range of values including that number plus or minus 10% of that number. Similarly, “about” a range refers to a range of values including 10% less than the lowest listed number of the range up to 10% more than the highest number of the range.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

### Applications of Target-Independent Microbe Detection

Microbial contents of biological or biomedical samples, ecological or environmental samples, complex biological environmental samples, industrial microbial samples, and food samples are frequently either identified or quantified through culture dependent methods. Culturing a microorganism can depend on various factors including, but not limited to, pH, temperature, humidity, and nutrients. It is often a time-consuming and difficult process to determine the culturing conditions for an unknown or previously uncultured organism.

Many microorganisms currently cannot be cultured in the laboratory. A significant amount of microbial biodiversity is overlooked by cultivation-based methods. Methods and compositions of the present disclosure can be applied to genetic analysis of organisms in metagenomic samples, such as microbes or viruses that cannot be cultured in a laboratory environment and that inhabit a wide variety of environments. Non-limiting examples of metagenomic samples include biological samples including tissues, urine, sweat, saliva, sputum, and feces; the air and atmosphere; water samples from bodies of water such as ponds, lakes, seas, oceans, etc; ecological samples such as soil and dirt; and foodstuffs. Analysis of microbial content in various metagenomic samples is useful in applications including, but not limited to, medicine, forensics, environmental monitoring, and food science.

Individual microbes or a “microbial signature” or “microbial fingerprint” comprising a panel of microbes is identified in a biological or biomedical sample obtained from a subject, for example mammalian subjects such as a human or other animal. In some aspects, such information is used for medical applications or purposes. Often, identification comprises determining the presence or the absence of a microbial genus or species, or microbial genera or species with previously unidentified or uncommon genetic mutations, such as mutations that can confer antibiotic resistance to bacterial strains. Sometimes, identification comprises determining the levels of microbial DNA from one or more microbial species or one or more microbial genera. In some cases, a microbial signature or fingerprint indicates a level of microbial DNA of a particular genus or species that is increased or significantly higher compared to the level of microbial DNA from a different genera or species in a sample. The microbial signature or fingerprint of a sample often indicates a level of microbial DNA from a particular genus or species that is decreased or significantly lower compared to the level of microbial DNA from other genera or species in the sample. A microbial signature or fingerprint of a sample is sometimes determined by quantifying the levels of microbial DNA of various types of microbes (e.g., different genera or species) that are present in the sample. The levels of microbial DNA of various genera or species of microbes that are present in a sample is often determined and compared to that of a control sample or standard.

The presence of a microbial genera or species in a subject suspected of having a medical condition, in some instances, is confidently diagnosed as having a medical condition being caused by the microbial genera or species. Optionally, this information is used to quarantine an individual from other individuals if the microbial genera or species is suspected of being transmittable to other individuals, for example by contact or proximity. In some cases, information regarding the microbe or microbial species present in a sample is used to determine a particular medical treatment to eliminate the microbe in the subject and treat, for example, a bacterial infection.

When the level of microbial DNA of a particular genus or species in a sample is decreased or significantly lower than a control sample or standard, the subject from which the sample was obtained is sometimes diagnosed as suffering from a disease, such as for example cancer (e.g., breast cancer). Often, the levels of microbial DNA of various genera or species of microbes that are present in a sample is determined and compared between the other various genera or species present in the sample. When the level of microbial DNA of a particular genus or species in a sample is decreased or significantly lower than the microbial DNA of other microbial genera or species detected in the sample, the subject from which the sample was obtained is likely suffering from a disease, such as for example cancer.

Individual microbes or a “microbial signature” or “microbial fingerprint” comprising a panel of microbes are identified in environmental or ecological samples, for example air samples, water samples, and soil or dirt samples. Identification of microbes and analysis of microbial diversity in environmental or ecological samples is often used to improve strategies for monitoring the impact of pollutants on ecosystems and for cleaning up contaminated environments. Increased understanding of how microbial communities cope with pollutants improves assessments of the potential of contaminated sites to recover from pollution and increases the chances of bioaugmentation or biostimulation. Such information provides valuable insights into the functional ecology of environmental communities. Microbial analysis is also used more broadly in some cases to identify species present the air, specific bodies of water, and samples of soil and dirt. This can, for example, be used to establish the range of invasive species and endangered species, and track seasonal populations.

Identification and analysis of microbial communities in environmental or ecological samples are also useful for agricultural applications. Microbial consortia perform a wide variety of ecosystem services necessary for plant growth, including fixing atmospheric nitrogen, nutrient cycling, suppressing disease, and sequestering iron and other metals. Such information is useful, for example to improve disease detection in crops and livestock and the adaptation of enhanced farming practices which improve crop health by harnessing the relationship between microbes and plants.

Individual microbes or a “microbial signature” or “microbial fingerprint” comprising a panel of microbes are sometimes identified in industrial samples of microbes, for example microbial communities used to produce various biologically active chemicals, such as fine chemicals, agrochemicals, and pharmaceuticals. Microbial communities produce a vast array of biologically active chemicals.

Microbial detection and identification based on sequence analysis are also useful for food safety, food authenticity, and fraud detection. For example, microbial detection and identification in metagenomic samples allow for detection and identification of nonculturable and previously unknown pathogens, including bacteria, viruses and parasites, in foods suspected of spoilage or contamination. With estimates that around 80 percent of foodborne disease cases in the U.S. are caused by unspecified agents, including known agents not yet recognized as causing foodborne illness, substances known to be in food but of unproven pathogenicity, and unknown agents, microbial analysis of entire populations can provide opportunities to reduce foodborne illnesses. With increasing awareness of the global supply of food and increasing awareness of sustainable practices in procuring foods such as seafood and shellfish, microbial detection cis useful to assess the authenticity of foods, for example determining if fish claiming to be from a particular region of the world is truly from that region of the world.

De novo genomic assembly can be improved by incorporating long range DNA interaction data obtained by linking together distant DNA sequences. One method to form these linkages is to assemble chromatin in vitro with genomic DNA and proteins such as histones. The assembled chromatin can then be cross-linked to fix long range interactions, and the sequence of DNA found within each is identified. One way to identify DNA sequences in an aggregate is to digest and re-ligate DNA, followed by identification of non-contiguous DNA sequences via sequencing. This approach, however, is limited by its capacity to identify only one pair of DNA sequences with an aggregate.

The present disclosure provides robust, cost-effective, and sample-efficient methods for producing long range sequence information, such as physical linkage information for assembled contigs that are bound by repetitive, hard to assemble sequence regions. The methods disclosed herein address previous shortcomings while producing sequence information or physical linkage information over comparatively vast genomic distances (up to megabases) due to the stabilization offered by chromatin and cross-linking. Furthermore, the methods disclosed herein may be realized with numerous distinct platforms, each with strengths and weaknesses for particular applications or targeted outcomes.

The present disclosure provides methods for identifying DNA sequences within a chromatin aggregate by associating the ends of DNA with reverse complementary sequences. In some cases, the chromatin is digested with a restriction enzyme, which may leave short overhangs of the restriction enzyme recognition site, and the DNA may be captured on a chromatin capture platform (e.g. a DNA microarray) using a sequence that is the reverse complement of the restriction enzyme recognition site. In further examples, a common adaptor is attached to the ends of DNA in the digested chromatin aggregate to allow for more powerful and flexible approaches towards analyzing the identity of DNA within individual aggregates. There are several advantages of attaching an adaptor to the chromatin aggregates. The capture sequence for hybridizing to the chromatin aggregate can be standardized across chromatin preparations and platforms. For instance, only one platform design may be needed and different methods for fragmenting the chromatin (e.g. using different restriction enzymes) may be employed. The adaptor may also be designed to comprise a unique barcode, which may allow for efficient use of the bandwidth of the chromatin capture platform. For example, several distinct in vitro chromatin assemblies may be prepared and attached to an adaptor with a unique barcode, so that the distinct chromatin assemblies may be applied to a same chromatin capture platform and still be identified in downstream analysis based on the barcode sequence. Alternatively, one in vitro chromatin assembly may be prepared and split into multiple aliquots, which are each linked to a chromatin adaptor with a unique barcode. In some cases, the aliquots are mixed backed together and applied to a same chromatin capture platform, but the distinct chromatin aggregates that share the same barcode can still be identified. The use of the adaptors may also negate the bias caused by digesting the chromatin aggregate with a restriction enzyme that has a non-palindromic recognition site, wherein only one of the single-strand overhangs is captured on the chromatin capture platform. In some cases, the adaptors are designed such that the capture sequence(s) has a desired melting temperature (Tm). In further examples, a protective moiety (e.g. a phosphorothiorate bond) is introduced into the adaptor to protect the short overhangs on the chromatin from exonuclease activity.

### Applications of Linkage Determination in a Heterologous Sample

Applications of the methods herein also relate to linkage determination for known or unknown molecules in a heterogeneous sample. Also contemplated herein are applications related to determination of linkage information in heterogeneous samples aside from novel organism detection. Often, linkage information is determined for nucleic acids such as chromosomes in a heterogeneous nucleic acid sample. A sample comprising DNA from a plurality of individuals is obtained, such as a sample from a crime scene, a urinal or toilet, a battlefield, a sink or garbage waste. Nucleic acid sequence information is obtained, for example via shotgun sequencing, and linkage information is determined. Often, an individual's unique genomic information is not identified by a single locus but by a combination of loci such as single nucleotide polymorphisms (SNPs), insertions or deletions (in/dels) or point mutations or alleles that collectively represent a unique or substantially unique genetic combination of traits. In many cases, no individual trait is sufficient to identify a specific individual. However, using linkage information such as that made available through practice of the methods herein, one identifies not only the aggregate alleles present in a heterogeneous sample, as with shotgun or alternate high-throughput sequencing approaches available in the art, bit one also determines specific combinations of alleles present in specific molecules in the sample. Thus, one determines not simply specific alleles in the sample, but the combinations of these alleles on chromosomes as necessary to map the allele combinations to specific individuals for which genome information is available through a previously obtained genomic sequence or through sequence information available from relatives. Linkage information is also valuable in cases where a gene is known to exist in a heterogeneous sample, but its genomic context is unknown. For example, in some cases an individual is known to harbor a harmful infection that is resistant to an antibiotic treatment. Shotgun sequencing is likely to identify the antibiotic resistance gene. However, through practice of the methods herein, valuable information is gained regarding the genomic context of the antibiotic resistance gene. Thus, by identifying not only the antibiotic resistance gene but the genome of the organism in which it resides, one is able to identify alternate treatments to target the antibiotic resistance gene host in light of the remainder of its genomic information. For example, a metabolic pathway absent from the resistant microbe or vulnerable to a second antibiotic is targeted such that the resistant microbe is cleared despite being resistant to the antibiotic if first choice. Alternately, using more complete genomic information regarding the host of an antibiotic resistance gene in a patient, one determines whether the resistance gene arises from a ‘wild’ microbial organism, or whether it is likely to have arisen from a laboratory strain of a microbe that ‘escaped’ from the laboratory or was intentionally released.

### Samples

A sample in which microbes are detected can be any sample comprising a microbial population or heterogeneous nucleic acid population. Examples include biological or biomedical samples from a human subject or animal subject; an environmental or ecological sample including but not limited to soil and water samples such as a water sample from a pond, lake, sea, ocean, or other source; or foodstuffs, such as those suspected of being spoiled or contaminated.

Biological samples can be obtained from a biological subject. A subject can refer to any organism (e.g., a eubacteria, archaea, viral organism, or eukaryote such as a plant, non-mammalian animal or mammal), including but not limited to humans, non-human primates, rodents, dogs, cats, pigs, fish, and the like. Samples can be obtained from any subject, individual, or biological source including, for example, human or non-human animals, including mammals and non-mammals, vertebrates and invertebrates. A sample can comprise an infected or contaminated tissue sample, such as for example a tissue sample comprising skin, heart, lung, kidney, breast, pancreas, liver, muscle, smooth muscle, bladder, gall bladder, colon, intestine, brain, prostate, esophagus, and thyroid. A sample can comprise an infected or contaminated biological sample, such as for example blood, urine, cerebrospinal fluid, seminal fluid, saliva, sputum, and stool.

Heterogeneous samples often comprise nucleic acids derived from at least two individuals, such as a sample obtained from a urinal or toilet used by two or more individuals, or a site where blood or tissue from at least two individuals is comingled such as a battlefield or a crime scene. Through the practice of methods disclosed herein, linkage information for the sample can be ascertained for known or unknown molecules in a heterogeneous sample.

Methods for obtaining a sample can be selected for the appropriate sample type and desired application. For example, a tissue sample may be obtained by biopsy or resection during a surgical procedure; blood may be obtained by venipuncture; and saliva, sputum, and stool can be self-provided by an individual in a receptacle.

A stool sample is often derived from an animal such as a mammal (e.g., non-human primate, equine, bovine, canine, feline, porcine and human). A stool sample can be of any suitable weight. A stool sample can be at least 50 g, 60 g, 70 g, 80 g, 90 g, 100 g, 110 g, 120 g, 130 g, 140 g, 150 g or more. A stool sample can contain water. In some aspects, a stool sample contains at least 60%, 65%, 70%, 75%, 80%, 85%, or 90% or more that 90% of water. Sometimes, a stool sample is stored. Stool samples can be stored for several days (e.g. between 3-5 days) at 2-8° C., or for longer periods of time (e.g. more than 5 days) at temperatures at −20° C. or lower. Often, a stool sample is provided by an individual or subject. Alternatively, a stool sample is collected from a place where stool is deposited. A stool sample sometimes comprises multiple samples collected from a single individual over a predetermined period of time. Stool samples collected over a period of time at multiple time-points are often used to monitor the biodiversity in the stool of an individual, for example during the course of treatment for an infection. Alternatively, a stool sample comprises samples from several individuals, for example several individuals suspected of being infected with the same pathogen or to have contracted the same disease.

Some samples comprise environmental or ecological samples comprising a microbial population or community. Non-limiting examples of environmental samples include atmosphere or air samples, soil or dirt samples, and water samples. Air samples can be analyzed to determine the microbial composition of air, for example air in areas that are suspected of harboring microbes considered health threats, for example, viruses causing illnesses. Often, understanding the microbial make-up of an air sample can be used to monitor changes in the environment.

Water samples are sometimes be analyzed for purposes including but not limited to public safety and environmental monitoring. Water samples, such as from a drinking water supply reservoir, can be analyzed to determine the microbial diversity in the drinking water supply and potential impact on human health. Water samples can be analyzed to determine the impact on microbial environments resulting from changes in local temperatures and compositions of gases in the atmosphere. Water samples, for example water sample from a pond, lake, sea, ocean, or other water body, can be sampled at various times of the year. Multiple samples are often acquired at various times of the year. Water samples can be collected at various depths from the surface of the body of water. For example, a water sample can be collected at the surface or at least 1 meter (e.g. at least 2, 3, 4, 5, 6, 7, 8, 9 meters or farther) from the surface of the body of water. In some instances, the water sample is collected from the floor of the body of water.

Soil and dirt samples are often sampled to study microbial diversity. Soil samples sometimes provide information regarding movement of viruses and bacteria in soils and waters and are often useful in bioremediation, in which genetic engineering can be applied to develop soil microbes capable of degrading hazardous pollutants. Soil microbial communities often harbor thousands of different organisms that contain a substantial number of genetic information, for example ranging from 2,000 to 18,000 different genomes estimated in one gram of soil. A soil sample is collected at various depths from the surface. Sometimes, soil is collected at the surface. Alternatively, soil is collected at least 1 in (e.g. at least 2, 3, 4, 5, 6, 7, 8, 9 or 10 in or farther) below the surface. For instance, soil is collected at depths between 1-10 in (e.g. between 2-9 in, 3-8 in, 4-7 in, or 5-6 in) below the surface. A soil sample can be collected at various times during the year. In some instances, a soil sample is collected in a specific season, such as winter, spring, summer or fall. Sometimes, a soil sample is collected in a particular month. Alternatively, a soil sample is collected after an environmental phenomenon, including but not limited to a tornado, hurricane, or thunderstorm. Multiple soil samples are often collected over a period of time to allow for monitoring of microbial diversity over a time course. A soil sample is often collected from various ecosystems, such as agroecosystems, forest ecosystems, and ecosystems from various geographical regions.

A food sample is contemplated to be any foodstuff suspected of contamination, spoilage, a cause of human illness or otherwise suspected of harboring a microbe or nucleic acid of interest. A food sample can be produced on a small scale, such as in a single shop. A food sample can be produced on an industrial scale, such as in a large food manufacturing or food processing plant. Examples of food samples without limitation include animal products including raw or cooked seafood, shellfish, raw or cooked eggs, undercooked meats including beef, pork, and poultry, unpasteurized milk, unpasteurized soft cheeses, raw hot dogs, and deli meats; plant products including fresh produce and salads; fruit products such as fresh produce and fruit juice; and processed and/or prepared foods such as home-made canned goods, mass-manufactured canned goods, and sandwiches. A food sample for analysis, such as a food sample suspected of being contaminated or spoiled, has often been stored at room temperature, for example between 20° C. and 25° C. For example, a food sample was stored at a temperature less than room temperature, such as a temperature less than 20° C., 18° C., 16° C., 14° C., 12° C., 10° C., 8° C., 6° C., 4° C., 2° C., 0° C., −10° C., −20° C., −40° C., −60° C., or −80° C. or lower. Alternatively, a food sample was stored at a temperature greater than room temperature, such as a temperature greater than 26° C., 28° C., 30° C., 32° C., 34° C., 36° C., 38° C., 40° C., or 50° C. or higher. Sometimes, a food sample was stored at an unknown temperature. A food sample has often been stored for a certain period of time, such as for example 1 day, 1 week, 1 month or 1 year. For example, a food sample was stored for at least 1 day, 1 week, 1 month, 6 months, 1 year, 2 years or longer. A food sample is often perishable and have a limited shelf life. A food sample produced in a manufacturing plant is sometimes obtained from a particular production lot or production period. Food samples are often obtained from different stores in different communities and from different manufacturing plants.

### Nucleic Acid Molecules

Nucleic acid molecules (e.g., DNA or RNA) can be isolated from a metagenomic sample containing a variety of other components, such as proteins, lipids and non-template nucleic acids. Nucleic acid molecules can be obtained from any cellular material, obtained from an animal, plant, bacterium, fungus, or any other cellular organism. Biological samples for use in the present disclosure also include viral particles or preparations. Nucleic acid molecules may be obtained directly from an organism or from a biological sample obtained from an organism, e.g., from blood, urine, cerebrospinal fluid, seminal fluid, saliva, sputum, stool and tissue. Nucleic acid molecules may be obtained directly from an ecological or environmental sample obtained from an organism, e.g., from an air sample, a water sample, and soil sample. Nucleic acid template may be obtained directly from food sample suspected of being spoiled or contaminated, e.g., a meat sample, a produce sample, a fruit sample, a raw food sample, a processed food sample, a frozen sample, etc.

Nucleic acids are extracted and purified using various methods. For example, nucleic acids are purified by organic extraction with phenol, phenol/chloroform/isoamyl alcohol, or similar formulations, including TRIzol and TriReagent. Non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., 1988), such precipitation methods being typically referred to as “salting-out” methods. Nucleic acid isolation and/or purification may comprise the use of magnetic particles to which nucleic acids can specifically or non-specifically bind, followed by isolation of the beads using a magnet, and washing and eluting the nucleic acids from the beads (see e.g. U.S. Pat. No. 5,705,628). The above isolation methods can be preceded by an enzyme digestion step to help eliminate unwanted protein from the sample, e.g., digestion with proteinase K, or other like proteases. See, e.g., U.S. Pat. No. 7,001,724. If desired, RNase inhibitors may be added to the lysis buffer. For certain cell or sample types, a protein denaturation/digestion step can be added to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can be generated, for example, by purification based on size, sequence, or other physical or chemical characteristic. In addition to an initial nucleic isolation step, purification of nucleic acids can be performed after any step in the methods of the disclosure, such as to remove excess or unwanted reagents, reactants, or products. For example, when the detection of RNA-encoded genomes is contemplated, nucleic acid samples are treated with reverse transcriptase so that RNA molecules in a nucleic acid sample serve as templates for the synthesis of complementary DNA molecules. Often such a treatment facilitates downstream analysis of the nucleic acid sample.

Nucleic acid template molecules are contemplated to be obtained through a broad range of approaches, such as described in U.S. Patent Application Publication Number US2002/0190663, published Oct. 9, 2003, which is hereby incorporated by reference in its entirety. Nucleic acid molecules are variously obtained from a biological sample by a variety of techniques such as those described by Maniatis, et al., Molecular Cloning: A Laboratory Manual, Cold Spring Harbor, N.Y., pp. 280-281 (1982) and in more recent updates to the well-known laboratory resource. The nucleic acids may first be extracted from the biological samples and then cross-linked in vitro. Native association proteins (e.g., histones) can further be removed from the nucleic acids.

The methods disclosed herein are often applied to any high molecular weight double stranded DNA including, for example, DNA isolated from tissues, cell culture, bodily fluids, animal tissue, plant, bacteria, fungi, viruses, etc.

Each of the plurality of independent samples independently often comprise at least 1 ng, 2 ng, 5 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, 5 μg, 10 μg, 20 μg, 50 μg, 100 μg, 200 μg, 500 μg, or 1000 μg, or more of nucleic acid material. Similarly, each of the plurality of independent samples independently may comprise less than about 1 ng, 2 ng, 5 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, 5 μg, 10 μg, 20 μg, 50 μg, 100 μg, 200 μg, 500 μg, 1000 μg or more of nucleic acid.

Various methods for quantifying nucleic acids are available. Non-limiting examples of methods for quantifying nucleic acids include spectrophotometric analysis and measuring fluorescence intensity of dyes that bind to nucleic acids and selectively fluoresce when bound, such as for example Ethidium Bromide.

### Nucleic Acid Complexes

Nucleic acids comprising DNA from a metagenomic or otherwise heterogeneous sample or samples is often bound to association molecules or nucleic acid binding moieties to form nucleic acid complexes. Sometimes, nucleic acid complexes comprise nucleic acids bound to a plurality of association molecules or moieties, such as polypeptides; non-protein organic molecules; and nanoparticles. Binding agents bind to individual nucleic acids at single or at multiple points of contact, such that the segments at these points of contact are held together independent of their common phosphodiester backbone.

Binding a nucleic acid often comprises forming linkages, for example covalent linkages, between segments of a nucleic acid molecule. Linkages are formed between local, adjacent or distant segments of a nucleic acid molecule. Binding a nucleic acid to form a nucleic acid complex often comprises cross-linking a nucleic acid to an association molecule or moiety (herein also referred to as a nucleic acid binding molecule or moiety). Association molecules are contemplated to comprise amino acids, including but not limited to peptides and proteins such as DNA binding proteins. Exemplary DNA binding proteins include native chromatin constituents such as histone, for example Histones 2A, 2B, 3A, 3B, 4A, and 4B. Often, the plurality of nucleic acid binding moieties comprises reconstituted chromatin or in vitro assembled chromatin. Chromatin is sometimes reconstituted from DNA molecules that are about 150 kbp in length. Alternatively, chromatin is reconstituted from DNA molecules that are at least 50, 100, 125, 150, 200, 250 kbp or more in length. Some representative binding proteins comprise transcription factors or transposases. Non-protein organic molecules are also compatible with the disclosure herein, such as protamine, spermine, spermidine or other positively charged molecules. Some association molecules comprise nanoparticles, such as nanoparticles having a positively charged surface. A number of nanoparticle compositions are compatible with the disclosure herein. In some aspects, the nanoparticles comprise silicon, such as silicon coated with a positive coating so as to bind negatively charged nucleic acids. In some cases, the nanoparticle is a platinum-based nanoparticle. The nanoparticles can be magnetic, which may facilitate the isolation of the cross-linked sequence segments.

A nucleic acid is bound to an association molecule by various methods consistent with the disclosure herein. Often, a nucleic acid is cross-linked to an association molecule. Methods of crosslinking include ultraviolet irradiation, chemical and physical (e.g., optical) crosslinking. Non-limiting examples of chemical crosslinking agents include formaldehyde and psoralen (Solomon et al., Proc. Natl. Acad. Sci. USA 82:6470-6474, 1985; Solomon et al., Cell 53:937-947, 1988). Cross-linking is performed through any number of approaches known in the art, such as by adding a solution comprising about 2% formaldehyde to a mixture comprising the nucleic acid molecule and chromatin proteins, although other concentrations are also contemplated and consistent with the disclosure herein. Other non-limiting examples of agents that can be used for cross-linking DNA include, but are not limited to, mitomycin C, nitrogen mustard, melphalan, 1,3-butadiene diepoxide, cis diaminedichloroplatinum (II) and cyclophosphamide. Some cross-linking agents form cross-links that bridge relatively short distances-such as about 2 Å, 3 Å, 4 Å, or 5 Å, while other cross-linking agents from longer bridging links.

Nucleic acid complexes, for example nucleic acids bound to in vitro assembled chromatin (herein referred to as chromatin aggregates) are assembled ‘free’ or alternately are attached to a solid support, including but not limited to beads, for example magnetic beads.

The nucleic acid binding moiety is contemplated to be or to comprise a category of protein, such as histones that form chromatin. The chromatin is often reconstituted chromatin or native chromatin. The nucleic acid binding moiety is alternatively distributed on solid support such as a microarray, a slide, a chip, a microwell, a column, a tube, a particle or a bead. For example, the solid support is coated with streptavidin and/or avidin. In other examples, the solid support is coated with an antibody. Further, the solid support is often additionally or alternatively comprises a glass, metal, ceramic or polymeric material. In some cases, the solid support is a nucleic acid microarray (e.g. a DNA microarray). Alternatively, the solid support can be a paramagnetic bead.

Nucleic acid complexes are often contemplated to be existent in a sample rather than being assembled subsequent to or concurrent with extraction. Often, nucleic acid complexes in such situations comprise native nucleosomes or other native nucleic acid binding molecules complexed to nucleic acids of the sample.

An example of a nucleic acid binding moiety that forms a structure is reconstituted chromatin. An important benefit of a nucleic acid binding moiety scaffold such as reconstituted chromatin is that it preserves physical linkage information of its constituent nucleic acids independent of their phosphodiester bonds. Accordingly, nucleic acids held together by reconstituted chromatin, optionally crosslinked to maintain stability, will maintain their proximity even if their phosphodiester bonds are broken, as may occur in internal labeling. Because of the reconstituted chromatin, the fragments will remain in proximity even though cleaved, thereby preserving phase or physical linkage information during an internal labeling process. Thus, when the exposed ends are re-ligated, they will ligate to segments derived from a common phase of a common molecule.

Nucleic acid complexes, either native or subsequently generated, are often independently stable. Alternatively, nucleic acid complexes, either native or subsequently generated, are stabilized by treatment with a cross-linking agent.

The DNA sample is often cross-linked to a plurality of association molecules. Sometimes, the association molecules comprise amino acids. Often, the association molecules comprise peptides or proteins. For example, some association molecules comprise histones. Alternatively, the association molecules comprise nanoparticles. The nanoparticle is often a platinum-based nanoparticle. Alternatively, the nanoparticle is a DNA intercalator, or any derivatives thereof. For example, the nanoparticle is a bisintercalator, or any derivatives thereof. Sometimes, the association molecules are from a different source than the first DNA molecule. The cross-linking is often conducted as part of a protocol as disclosed herein, or has alternatively been conducted previously. For example, previously fixed samples (e.g., formalin-fixed paraffin-embedded (FFPE)) samples are often processed and analyzed with techniques of the present disclosure.

### Chromatin Reconstitution

The assembly of nucleic acids onto a nucleic acid binding moiety for the preservation of phase information during cleavage and rearrangement of the nucleic acid molecule is often accomplished through the assembly of reconstituted chromatin onto a nucleic acid sample. Reconstituted chromatin as used herein is used broadly, ranging from reassembly of native chromatin constituents onto a nucleic acid, to binding of a nucleic acid to non-biological particles.

Reconstituted chromatin as a binding moiety is accomplished by a number of approaches. Reconstituted chromatin as contemplated herein is used broadly to encompass binding of a broad number of binding moieties to a naked nucleic acid. Binding moieties include histones and nucleosomes, but in some interpretations of reconstituted chromatin also other nuclear proteins such as transcription factors, transposons, or other DNA or other nucleic acid binding proteins, spermine or spermidine or other non-polypeptide nucleic acid binding moieties, nanoparticles such as organic or inorganic nanoparticle nucleic acid binding agents.

Reconstituted chromatin is often used in reference to the reassembly of native chromatin constituents or homologues of native chromatin constituents onto a naked nucleic acid, such as reassembly of histones or nucleosomes onto a native nucleic acid.

Two approaches to reconstitute chromatin include (1) ATP-independent random deposition of histones onto DNA, and (2) ATP-dependent assembly of periodic nucleosomes. This disclosure contemplates the use of either approach with one or more methods disclosed herein. Examples of both approaches to generate chromatin can be found in Lusser et al. (“Strategies for the reconstitution of chromatin,” Nature Methods (2004), 1(1): 19-26), which is incorporated herein by reference in its entirety.

Other approaches to reconstituting chromatin, either strictly defined as nucleosome or histone addition to naked nucleic acids, or more broadly defined as the addition of any moiety to a naked nucleic acid, are contemplated herein, and neither the composition of chromatin nor the approach to its reconstitution should be considered limiting. In some cases, ‘chromatin reconstitution’ refers to the generation not of native chromatin but of generation of novel nucleic acid complexes, such as complexes comprising nucleic acids stabilized by binding to nanoparticles, such as nanoparticles having a surface comprising a moiety that facilitates nucleic acid binding or nucleic acid binding and cross-linking.

Alternately, no reconstitution is performed, and native nucleic acid complexes are relied upon to stabilize nucleic acids for downstream analysis. Often, such nucleic acid complexes comprise native histones, but complexes comprising other nuclear proteins, DNA binding proteins, transposases, topoisomerases, or other DNA binding proteins are contemplated.

Natural and non-natural chromatin analogs are contemplated. Nanoparticles, such as nanoparticles having a positively coated outer surface to facilitate nucleic acid binding, or a surface activatable for cross-linking to nucleic acids, or both a positively coated outer surface to facilitate nucleic acid binding and a surface activatable for cross-linking to nucleic acids, are contemplated herein. In some embodiments, nanoparticles comprise silicon.

Some methods disclosed herein are used with DNA associated with nanoparticles. Often, the nanoparticles are positively charged. For example, the nanoparticles are coated with amine groups, and/or amine-containing molecules. The DNA and the nanoparticles aggregate and condense, similar to native or reconstituted chromatin. Further, the nanoparticle-bound DNA is induced to aggregate in a fashion that mimics the ordered arrays of biological nucleosomes (i.e. chromatin). The nanoparticle-based method can be less expensive, faster to assemble, provides a better recovery rate than using reconstituted chromatin, and/or allows for reduced DNA input requirements.

A number of factors can be varied to influence the extent and form of condensation including the concentration of nanoparticles in solution, the ratio of nanoparticles to DNA, and the size of nanoparticles used. In some cases, the nanoparticles are added to the DNA at a concentration greater than about 1 ng/mL, 2 ng/mL, 3 ng/mL, 4 ng/mL, 5 ng/ml, 6 ng/mL, 7 ng/mL, 8 ng/mL, 9 ng/mL, 10 ng/mL, 15 ng/ml, 20 ng/ml, 25 ng/ml, 30 ng/ml, 40 ng/mL, 50 ng/mL, 60 ng/mL, 70 ng/mL, 80 ng/mL, 90 ng/mL, 100 ng/ml, 120 ng/ml, 140 ng/mL, 160 ng/mL, 180 ng/mL, 200 ng/mL, 250 ng/ml, 300 ng/mL, 400 ng/mL, 500 ng/mL, 600 ng/mL, 700 ng/mL, 800 ng/ml, 900 ng/ml, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, 20 μg/mL, 25 μg/mL, 30 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 250 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, or 100 mg/mL. In some cases, the nanoparticles are added to the DNA at a concentration less than about 1 ng/mL, 2 ng/ml, 3 ng/mL, 4 ng/mL, 5 ng/mL, 6 ng/mL, 7 ng/mL, 8 ng/ml, 9 ng/mL, 10 ng/ml, 15 ng/mL, 20 ng/mL, 25 ng/ml, 30 ng/mL, 40 ng/ml, 50 ng/mL, 60 ng/ml, 70 ng/ml, 80 ng/mL, 90 ng/mL, 100 ng/mL, 120 ng/mL, 140 ng/mL, 160 ng/mL, 180 ng/ml, 200 ng/mL, 250 ng/ml, 300 ng/mL, 400 ng/mL, 500 ng/mL, 600 ng/mL, 700 ng/ml, 800 ng/mL, 900 ng/mL, 1 μg/mL, 2 μg/mL, 3 μg/mL, 4 μg/mL, 5 μg/mL, 6 μg/mL, 7 μg/mL, 8 μg/mL, 9 μg/mL, 10 μg/mL, 15 μg/mL, 20 μg/mL, 25 μg/mL, 30 μg/mL, 40 μg/mL, 50 μg/mL, 60 μg/mL, 70 μg/mL, 80 μg/mL, 90 μg/mL, 100 μg/mL, 120 μg/mL, 140 μg/mL, 160 μg/mL, 180 μg/mL, 200 μg/mL, 250 μg/mL, 300 μg/mL, 400 μg/mL, 500 μg/mL, 600 μg/mL, 700 μg/mL, 800 μg/mL, 900 μg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, or 100 mg/mL. In some cases, the nanoparticles are added to the DNA at a weight-to-weight (w/w) ratio greater than about 1:10000, 1:5000, 1:2000, 1:1000, 1:500, 1:200, 1:100, 1:50, 1:20, 1:10, 1:5, 1:2, 1:1, 2:1, 5:1, 10:1, 20:1, 50:1, 100:1, 200:1, 500:1, 1000:1, 2000:1, 5000:1, or 10000:1. In some cases, the nanoparticles are added to the DNA at a weight-to-weight (w/w) ratio less than about 1:10000, 1:5000, 1:2000, 1:1000, 1:500, 1:200, 1:100, 1:50, 1:20, 1:10, 1:5, 1:2, 1:1, 2:1, 5:1, 10:1, 20:1, 50:1, 100:1, 200:1, 500:1, 1000:1, 2000:1, 5000:1, or 10000:1. In some cases, the nanoparticles have a diameter greater than about 1 nm 1 nm, 2 nm, 3 nm, 4 nm, 5 nm, 6 nm, 7 nm, 8 nm, 9 nm, 10 nm, 15 nm, 20 nm, 25 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 15 μm, 20 μm, 25 μm, 30 μm, 40 μm, 50 μm, 60 μm, 70 μm, 80 μm, 90 μm, or 100 μm. In some cases, the nanoparticles have a diameter less than about 1 nm 1 nm, 2 nm, 3 nm, 4 nm, 5 nm, 6 nm, 7 nm, 8 nm, 9 nm, 10 nm, 15 nm, 20 nm, 25 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, 600 nm, 700 nm, 800 nm, 900 nm, 1 μm, 2 μm, 3 μm, 4 μm, 5 μm, 6 μm, 7 μm, 8 μm, 9 μm, 10 μm, 15 μm, 20 μm, 25 μm, 30 μm, 40 μm, 50 μm, 60 μm, 70 μm, 80 μm, 90 μm, or 100 μm.

Furthermore, the nanoparticles may be immobilized on solid substrates (e.g. beads, slides, or tube walls) by applying magnetic fields (in the case of paramagnetic nanoparticles) or by covalent attachment (e.g. by cross-linking to poly-lysine coated substrate). Immobilization of the nanoparticles may improve the ligation efficiency thereby increasing the number of desired products (signal) relative to undesired (noise).

Reconstituted chromatin is optionally contacted to a crosslinking agent such as formaldehyde to further stabilize the DNA-chromatin complex.

Reconstituted chromatin is differentiated from chromatin formed within a cell/organism over various features. First, reconstituted chromatin is often generated from isolated naked DNA. For many samples, the collection of naked DNA samples is achieved by using any one of a variety of noninvasive to invasive methods, such as by collecting bodily fluids, swabbing buccal or rectal areas, taking epithelial samples, etc. These approaches are generally easier, faster, and less expensive than isolation of native chromatin.

Second, reconstituting chromatin substantially reduces the formation of inter-chromosomal and other long-range interactions that generate artifacts for genome assembly and haplotype phasing. Often, a sample has less than about 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.5, 0.4, 0.3, 0.2, 0.1, 0.01, 0.001% or less inter-chromosomal or intermolecular crosslinking according to the methods and compositions of the disclosure. In some examples, the sample has less than about 30% inter-chromosomal or intermolecular crosslinking. In some examples, the sample has less than about 25% inter-chromosomal or intermolecular crosslinking. In some examples, the sample has less than about 20% inter-chromosomal or intermolecular crosslinking. In some examples, the sample has less than about 15% inter-chromosomal or intermolecular crosslinking. In some examples, the sample has less than about 10% inter-chromosomal or intermolecular crosslinking. In some examples, the sample has less than about 5% inter-chromosomal or intermolecular crosslinking. In some examples, the sample may have less than about 3% inter-chromosomal or intermolecular crosslinking. In further examples, may have less than about 1% inter-chromosomal or intermolecular crosslinking. As inter-chromosomal interactions represent interactions between molecular sections that are not in phase, their reduction or elimination is beneficial to some goals of the present disclosure, that is, the efficient, rapid assembly of phased nucleic acid information.

Third, the frequency of sites that are capable of crosslinking and thus the frequency of intramolecular crosslinks within the polynucleotide is adjustable. For example, the ratio of DNA to histones can be varied, such that the nucleosome density can be adjusted to a desired value. Often, the nucleosome density is reduced below the physiological level. Accordingly, the distribution of crosslinks can be altered to favor longer-range interactions. Alternatively, sub-samples with varying cross-linking density may be prepared to cover both short- and long-range associations.

For example, the crosslinking conditions can be adjusted such that at least about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 25%, about 30%, about 40%, about 45%, about 50%, about 60%, about 70%, about 80%, about 90%, about 95%, or about 100% of the crosslinks so as to join DNA segments that are at least about 50 kb, about 60 kb, about 70 kb, about 80 kb, about 90 kb, about 100 kb, about 110 kb, about 120 kb, about 130 kb, about 140 kb, about 150 kb, about 160 kb, about 180 kb, about 200 kb, about 250 kb, about 300 kb, about 350 kb, about 400 kb, about 450 kb, or about 500 kb apart on a sample DNA molecule.

### Cleaving Nucleic Acid Molecules

Nucleic acid molecules, such as bound nucleic acid molecules from a metagenomic sample in nucleic acid complexes, are often cleaved to expose internal nucleic acid ends and create double-stranded breaks. For example, a nucleic acid molecule, such as a nucleic acid molecule in a nucleic acid complex, is cleaved to expose nucleic acid ends and form at least two fragments or segments that are not physically linked at their phosphodiester backbone. Various methods are contemplated to be used to cleave internal nucleic acid ends and/or generate fragments derived from a nucleic acid, including but not limited to mechanical, chemical, and enzymatic methods such as shearing, sonication, nonspecific endonuclease treatment, or specific endonuclease treatment. Alternate approaches involve enzymatic cleavage, such as with a topoisomerase, a base-repair enzyme, a transpose such as Tn5, or a phosphodiester backbone nicking enzyme.

A nucleic acid is often cleaved by digesting. Digestion sometimes comprises contacting with a restriction endonuclease. Restriction endonucleases can be selected in light of known genomic sequence information to tailor an average number of free nucleic acid ends that result from digesting. Restriction endonucleases can cleave at or near specific recognition nucleotide sequences known as restriction sites. Restriction endonucleases having restriction sites with higher relative abundance throughout the genome can be used during digestion to produce a greater number of exposed nucleic acid ends compared to restriction endonucleases having restriction sites with lower relative abundance, as more restrictions sites can result in more cleaved sites. For example, restriction endonucleases with non-specific restriction sites, or more than one restriction site, are used. A non-limiting example of a non-specific restriction site is CCTNN. The bases A, C, G, and T refer to the four nucleotide bases of a DNA strand-adenine, cytosine, guanine, and thymine. The base N represents any of the four DNA bases-A, C, G, and T. Rather than recognizing a specific sequence for cleavage, an enzyme with the corresponding restriction site can recognize more than one sequence for cleavage. For example, the first five bases that are recognized can be CCTAA, CCTAT, CCTAG, CCTAC, CCTTA, CCTTT, CCTTG, CCTTC, CCTCA, CCTCT, CCTCG, CCTCC, CCTGA, CCTGT, CCTGG, or CCTGC (16 possibilities). Alternatively, use of an enzyme with a non-specific restriction site results in a larger number of cleavage sites compared to an enzyme with a specific restriction site. Restriction endonucleases can have restriction recognition sequences of at least 4, 5, 6, 7, 8 base pairs or longer. Restriction enzymes for digesting nucleic acid complexes can cleave single-stranded and/or double-stranded nucleic acids. Restriction endonucleases can produce single-stranded breaks or double-stranded breaks. Restriction endonuclease cleavage can produce blunt ends, 3′ overhangs, or 5′ overhangs. A 3′ overhang can be at least 1, 2, 3, 4, 5, 6, 7, 8, or 9 bases in length or longer. A 5′ overhang can be at least 1, 2, 3, 4, 5, 6, 7, 8, or 9 bases in length or longer. Examples of restriction enzymes include, but are not limited to, AatII, Acc65I, AccI, AciI, AclI, AcuI, AfeI, AflII, AflIII, AgeI, AhdI, AleI, AluI, AlwI, AlwNI, ApaI, ApaLI, ApeKI, ApoI, AscI, AseI, AsiSI, AvaI, AvalI, AvrII, BaeGI, BaeI, BamHI, BanI, BanII, BbsI, BbvCI, BbvI, BccI, BceAI, BcgI, BciVI, BclI, BfaI, BfuAI, BfuCI, BglI, BglII, BlpI, BmgBI, BmrI, BmtI, BpmI, BpulOI, BpuEI, BsaAI, BsaBI, BsaHI, BsaI, BsaJI, BsaWI, BsaXI, BscRI, BscYI, BsgI, BsiEI, BsiHKAI, BsiWI, BslI, BsmAI, BsmBI, BsmFI, BsmI, BsoBI, Bsp1286I, BspCNI, BspDI, BspEI, BspHI, BspMI, BspQI, BsrBI, BsrDI, BsrFI, BsrGI, BsrI, BssHII, BssKI, BssSI, BstAPI, BstBI, BstEII, BstNI, BstUI, BstXI, BstYI, BstZ17I, Bsu36I, BtgI, BtgZI, BtsCI, BtsI, Cac8I, ClaI, CspCI, CviAII, CviKI-1, CviQI, DdcI, DpnI, DpnII, Dral, DraIII, DrdI, EacI, EagI, EarI, EciI, Eco53kI, EcoNI, EcoO109I, EcoP15I, EcoRI, EcoRV, FatI, FauI, Fnu4HI, FokI, FseI, FspI, HaelI, HaelII, HgaI, HhaI, HincII, HindIII, HinfI, HinPII, HpaI, HpaII, HphI, Hpyl66II, Hpy 188I, Hpy 188III, Hpy991, Hpy AV, HpyCH4III, HpyCH4IV, HpyCH4V, KasI, KpnI, MboI, MboII, MfeI, MluI, MlyI, Mmel, MnlI, MscI, MseI, MslI, MspAlI, MspI, MwoI, NaeI, NarI, Nb.BbvCI, Nb.BsmI, Nb.BsrDI, Nb.BtsI, NciI, NcoI, NdeI, NgoMIV, NheI, NlaIII, NlaIV, NmeAIII, NotI, NruI, NsiI, NspI, Nt. AlwI, Nt.BbvCI, Nt.BsmAI, Nt.BspQI, Nt. BstNBI, Nt. CviPII, PacI, PaeR7I, PciI, PflFI, PflMI, PhoI, PleI, PmeI, PmlI, PpuMI, PshAI, PsiI, PspGI, PspOMI, PspXI, PstI, PvuI, PvulI, RsaI, RsrII, SacI, SacII, SalI, SapI, Sau3AI, Sau96I, SbfI, ScaI, ScrFI, SexAI, SfaNI, SfcI, SfiI, SfoI, SgrAI, SmaI, SmlI, SnaBI, SpeI, SphI, SspI, StuI, StyD4I, StyI, SwaI, T, TaqαI, TfiI, TliI, TseI, Tsp45I, Tsp509I, TspMI, TspRI, Tth111I, XbaI, XcmI, XhoI, XmaI, XmnI, and ZraI.

Alternatively, a combination of two or more isoschizomer enzymes (e.g., enzymes that cleave the same restriction sequence) are used. The isoschizomers often recognize and cleave a GATC sequence. For example, the isoschizomers can be BfuCI enzymes. The isoschizomers may be selected from MboI, DpnI, Sau3AI, and BfuCI. Alternatively, the two or more isoschizomers differ in their sensitivity to a base modification, such as methylation, hydroxymethylation, and oxidation. Methylation can be dam methylation, dcm methylation, or CpG methylation. Sensitivity to a base modification can be described as blocked, not blocked, or required. For example, a base modification can block the activity of a restriction enzyme or isoschizomer if the restriction enzyme or isoschizomer is not capable of cleaving a corresponding restriction sequence in the presence of the given base modification state, such as methylation. In other examples, a base modification cannot block the activity of a restriction enzyme or isoschizomer if the restriction enzyme or isoschizomer is capable of cleaving a corresponding restriction sequence in the presence of the given base modification state, such as methylation. In other examples, a base modification can be required for the activity of a restriction enzyme or isoschizomer if the restriction enzyme or isoschizomer is not capable of cleaving a corresponding restriction sequence in the absence of the given base modification state and is capable of cleaving a corresponding restriction sequence in the presence of the given base modification state.

A table of examples of isoschizomer sets wherein at least one member differs in its sensitivity to a modification is given below.

TABLE 1
Isoschizomer groups showing variation in sensitivity to modification

| Category | Enzyme | dam | dcm | CpG | Isoschizomers |
|---|---|---|---|---|---|
| methyl | DpnI | not | not | Blocked by | BfuCI, BscFI, Bsp143I, |
| dependent | | sensitive | sensitive | Overlapping | BssMI, BstENII, |
| | | | | | BstKTI, BstMBI, DpnII, |
| | | | | | Kzo9I, MalI, MboI, |
| | | | | | NdeII, Sau3AI |
| methyl | DpnII | Blocked | not | not | BfuCI, BscFI, Bsp143I, |
| sensitive | | | sensitive | sensitive | BssMI, BstENII, |
| | | | | | BstKTI, BstMBI, DpnI, |
| | | | | | Kzo9I, MalI, MboI, |
| | | | | | NdeII, Sau3AI |
| methyl | MboI | Blocked | not | Impaired by | BfuCI, BscFI, Bsp143I, |
| sensitive | | | sensitive | Overlapping | BssMI, BstENII, |
| | | | | | BstKTI, BstMBI, DpnI, |
| | | | | | DpnII, Kzo9I, MalI, |
| | | | | | NdeII, Sau3AI |
| methyl | HpaII | not | not | Blocked | MspI, BsiSI, HapII |
| sensitive | | sensitive | sensitive | | |
| methyl | ScrFI | not | Blocked by | Blocked by | Bme1390I, BmrFI, |
| sensitive | | sensitive | Overlapping | Overlapping | BssKI, BstSCI, MspR9I, |
| | | | | | StyD4I |
| methyl | Aat II | not | not | Blocked | ZraI |
| sensitive | | sensitive | sensitive | | |
| methyl | Acc II | not | not | Blocked | Bsh1236I, BspFNI, |
| sensitive | | sensitive | sensitive | | BstFNI, BstUI, FnuDII, |
| | | | | | MvnI, ThaI |
| methyl | Aor13H | Blocked by | not | Impaired | AccIII, BlfI, BseAI, |
| sensitive | I | Overlapping | sensitive | | BsiMI, Bsp13I, BspEI, |
| | | | | | BspMII, Kpn2I, MroI |
| methyl | Aor51H | not | not | Blocked | AfeI, Eco47III, FunI |
| sensitive | I | sensitive | sensitive | | |
| methyl | BspT10 | not | not | Blocked | AsuII, Bpu14I, BsiCI, |
| sensitive | 4 I | sensitive | sensitive | | Bsp119I, BstBI, Csp45I, |
| | | | | | NspV, SfuI |
| methyl | BssH II | not | not | Blocked | BsePI, PauI, PteI |
| sensitive | | sensitive | sensitive | | |
| methyl | Cfr10 I | not | not | Blocked | Bse118I, BsrFαI, BssAI |
| sensitive | | sensitive | sensitive | | |
| methyl | Cla I | Blocked by | not | Blocked | BanIII, Bsa29I, BseCI, |
| sensitive | | Overlapping | sensitive | | BshVI, BsiXI, Bsp106I, |
| | | | | | BspDI, BspXI, Bsu15I, |
| | | | | | BsuTUI, ZhoI |
| methyl | CpoI | | | | CspI, Rsr2I, RsrII |
| sensitive | | | | | |
| methyl | RsrII | not | not | Blocked | CpoI, CspI, Rsr2I |
| sensitive | | sensitive | sensitive | | |
| methyl | Eco52 I | not | not | Blocked | BseX3I, BstZI, EagI, |
| sensitive | | sensitive | sensitive | | EclXI, XmaIII |
| methyl | Hae II | not | not | Blocked | BfoI, Bsp143II, BstH2I |
| sensitive | | sensitive | sensitive | | |
| methyl | Hha I | not | not | Blocked | AspLEI, BstHHI, CfoI, |
| sensitive | | sensitive | sensitive | | GlaI, R9529, HinP1I, |
| | | | | | HspAI |
| methyl | Nae I | not | not | Blocked | KroI, MroNI, NgoAIV, |
| sensitive | | sensitive | sensitive | | NgoMIV, PdiI |
| methyl | Not I | not | not | Blocked | CciNI |
| sensitive | | sensitive | sensitive | | |
| methyl | Nru I | Blocked by | not | Blocked | Bsp68I, BtuMI, RruI |
| sensitive | | Overlapping | sensitive | | |
| methyl | Nsb I | not | not | Blocked | Acc16I, AviII, FspI, |
| sensitive | | sensitive | sensitive | | MstI |
| methyl | PmaC I | not | not | Blocked | AcvI, BbrPI, Eco72I, |
| sensitive | | sensitive | sensitive | | PmlI, PspCI |
| methyl | Psp1406 | not | not | Blocked | AclI |
| sensitive | I | sensitive | sensitive | | |
| methyl | Pvu I | not | not | Blocked | BpvUI, BspCI, MvrI, |
| sensitive | | sensitive | sensitive | | Ple19I |
| methyl | Sac II | not | not | Blocked | Cfr42I, KspI, Sfr303I, |
| sensitive | | sensitive | sensitive | | SgrBI, SstII |
| methyl | Sma I | not | not | Blocked | Cfr9I, PspAI, TspMI, |
| sensitive | | sensitive | sensitive | | XmaCI |
| methyl | SnaB I | not | not | Blocked | BstSNI, Eco105I |
| sensitive | | sensitive | sensitive | | |

In some cases where at least two restriction enzymes are used, at least one restriction enzyme is not an isoschizomer of at least one other restriction enzyme.

Optionally, two restriction enzymes or isoschizomers with differing sensitivities to a base modification are used. In some examples, three restriction enzymes or isoschizomers with differing sensitivities to a base modification are used. In some examples, four restriction enzymes or isoschizomers with differing sensitivities to a base modification are used. In some examples, more than four restriction enzymes or isoschizomers with differing sensitivities to a base modification are used.

Where two or more restriction enzymes or isoschizomers are used, the two or more restriction enzymes or isoschizomers are optionally used in a single restriction reaction. In some cases, the two or more restriction enzymes or isoschizomers are used in a separate restriction reactions. The separate restriction reactions can be performed in parallel or sequentially.

When a restriction enzyme or isoschizomer is used on a sample in a modification state in which the restriction enzyme or isoschizomer activity is blocked, then the sample will not be cut by that restriction enzyme or isoschizomer. Likewise, if a restriction enzyme or isoschizomer is used on a heterogeneous sample, wherein a fraction of the sample is in a modification state in which the restriction enzyme or isoschizomer activity is blocked, then said fraction of the sample will not be cut by that restriction enzyme or isoschizomer. Thus, downstream read pairs generated from ligated free ends will not include sequence from samples that were not able to be cleaved by the selected restriction enzyme or isoschizomer.

Alternative cleavage approaches are also consistent with the disclosure herein. For example, a transposase is optionally used in combination with unlinked left and right border oligonucleic acid molecules so as to create a sequence-independent break in a nucleic acid that is marked by the attachment of the transposase-delivered oligonucleic acid molecules. The oligonucleic acid molecules are synthesized in some cases to comprise punctuation-compatible overhangs, or to be compatible with one another, such that the oligonucleic acid molecules are ligated to one another and serve as the punctuation molecules. A benefit of this type of alternative approach is that cleavage is sequence independent, and thus more likely to vary from one copy of a nucleic acid to another, even if the sequence of two nucleic acid molecules is locally identical.

Often, the exposed nucleic acid ends are desirably sticky ends, for example as results from contacting to a restriction endonuclease. For example, a restriction endonuclease is used to cleave a predictable overhang, followed by ligation with a nucleic acid end (such as a punctuation oligonucleotide) comprising an overhang complementary to the predictable overhang on a DNA fragment. Optionally, the 5′ and/or 3′ end of a restriction endonuclease-generated overhang is partially filled in. Alternatively, the overhang is filled in with a single nucleotide.

DNA fragments having an overhang are often joined to one or more nucleic acids, such as punctuation oligonucleotides, oligonucleotides, adapter oligonucleotides, or polynucleotides, having a complementary overhang, such as in a ligation reaction. For example, a single adenine is added to the 3′ ends of end repaired DNA fragments using a template independent polymerase, followed by ligation to one or more punctuation oligonucleotides each having a thymine at a 3′ end. Alternatively, nucleic acids, such as oligonucleotides or polynucleotides are joined to blunt end double-stranded DNA molecules which have been modified by extension of the 3′ end with one or more nucleotides followed by 5′ phosphorylation. Sometimes, extension of the 3′ end is performed with a polymerase such as, Klenow polymerase or any of the suitable polymerases provided herein, or by use of a terminal deoxynucleotide transferase, in the presence of one or more dNTPs in a suitable buffer that contains magnesium. Often, target polynucleotides having blunt ends are joined to one or more adapters comprising a blunt end. Phosphorylation of 5′ ends of DNA fragment molecules may be performed for example with T4 polynucleotide kinase in a suitable buffer containing ATP and magnesium. The fragmented DNA molecules may optionally be treated to dephosphorylate 5′ ends or 3′ ends, for example, by using enzymes known in the art, such as phosphatases.

### Ligation

Cleaved nucleic acid molecules can be ligated by proximity ligation using various methods. Ligation of cleaved nucleic acid molecules can be accomplished by enzymatic and non-enzymatic protocols. Examples of ligation reactions that are non-enzymatic can include the non-enzymatic ligation techniques described in U.S. Pat. Nos. 5,780,613 and 5,476,930, each of which is herein incorporated by reference in its entirety. Enzymatic ligation reactions can comprise use of a ligase enzyme. Non-limiting examples of ligase enzymes are ATP-dependent double-stranded polynucleotide ligases, NAD+ dependent DNA or RNA ligases, and single-strand polynucleotide ligases. Non-limiting examples of ligases are Escherichia coli DNA ligase, Thermus filiformis DNA ligase, Tth DNA ligase, Thermus scotoductus DNA ligase (I and II), T3 DNA ligase, T4 DNA ligase, T4 RNA ligase, T7 DNA ligase, Taq ligase, Ampligase (Epicentre®Technologies Corp.), VanC-type ligase, 9° N DNA Ligase, Tsp DNA ligase, DNA ligase I, DNA ligase III, DNA ligase IV, Sso7-T3 DNA ligase, Sso7-T4 DNA ligase, Sso7-T7 DNA ligase, Sso7-Taq DNA ligase, Sso7-E. coli DNA ligase, Sso7-Ampligase DNA ligase, and thermostable ligases. Ligase enzymes may be wild-type, mutant isoforms, and genetically engineered variants. Ligation reactions can contain a buffer component, small molecule ligation enhancers, and other reaction components.

Punctuation oligonucleotides are optionally utilized in connecting exposed cleaved ends. A punctuation oligonucleotide includes any oligonucleotide that can be joined to a target polynucleotide, so as to bridge two cleaved internal ends of a sample molecule undergoing phase-preserving rearrangement. Punctuation oligonucleotides can comprise DNA, RNA, nucleotide analogues, non-canonical nucleotides, labeled nucleotides, modified nucleotides, or combinations thereof. In many examples, double-stranded punctuation oligonucleotides comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. Optionally, different punctuation oligonucleotides are joined to target polynucleotides in sequential reactions or simultaneously. For example, the first and second punctuation oligonucleotides can be added to the same reaction. Alternately, punctuation oligo populations are uniform.

Punctuation oligonucleotides can be manipulated prior to combining with target polynucleotides. For example, terminal phosphates can be removed. Such a modification precludes location of punctuation oligos to one another rather than to cleaved internal ends of a sample molecule.

Punctuation oligonucleotides contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different punctuation oligonucleotides or subsets of different punctuation oligonucleotides, one or more restriction enzyme recognition sites, one or more overhangs complementary to one or more target polynucleotide overhangs, one or more probe binding sites, one or more random or near-random sequences, and combinations thereof. In some examples, two or more sequence elements are non-adjacent to one another (e.g. separated by one or more nucleotides), adjacent to one another, partially overlapping, or completely overlapping. For example, an amplification primer annealing sequence also serves as a sequencing primer annealing sequence. In certain instances, sequence elements are located at or near the 3′ end, at or near the 5′ end, or in the interior of the punctuation oligonucleotide.

In alternate embodiments, the punctuation oligo comprises a minimal complement of bases to maintain integrity of the double-stranded molecule, so as to minimize the amount of sequence information it occupies in a sequencing reaction, or the punctuation oligo comprises an optimal number of bases for ligation, or the punctuation oligo length is arbitrarily determined.

Often, a punctuation oligonucleotide comprises a 5′ overhang, a 3′ overhang, or both that is complementary to one or more target polynucleotides. In certain instances, complementary overhangs are one or more nucleotides in length, including but not limited to 1, 2, 3, 4, 5, 6, 7, 8, 9, 10, 11, 12, 13, 14, 15, or more nucleotides in length. For example, the complementary overhang is about 1, 2, 3, 4, 5 or 6 nucleotides in length. Sometimes, a punctuation oligonucleotide overhang is complementary to a target polynucleotide overhang produced by restriction endonuclease digestion or other DNA cleavage method.

Punctuation oligonucleotides are contemplated to have any suitable length, at least sufficient to accommodate the one or more sequence elements of which they are comprised. In some embodiments, punctuation oligonucleotides are about, less than about, or more than about 4, 5, 6, 7, 8, 9, 10, 15, 20, 25, 30, 35, 40, 45, 50, 55, 60, 65, 70, 75, 80, 90, 100, 200, or more nucleotides in length. In some examples, the punctuation oligonucleotide is 5 to 15 nucleotides in length. In further examples, the punctuation oligonucleotide is about 20 to about 40 nucleotides in length.

Preferably, punctuation oligonucleotides are modified, for example by 5′ phosphate excision (via calf alkaline phosphatase treatment, or de novo by synthesis in the absence of such moieties), so that they do not ligate with one another to form multimers. 3′ OH (hydroxyl) moieties are able to ligate to 5′ phosphates on the cleaved nucleic acids, thereby supporting ligation to a first or a second nucleic acid segment.

An adapter includes any oligonucleotide having a sequence that can be joined to a target polynucleotide. In various examples, adapter oligonucleotides comprise DNA, RNA, nucleotide analogues, non-canonical nucleotides, labeled nucleotides, modified nucleotides, or combinations thereof. For example, adapter oligonucleotides are single-stranded, double-stranded, or partial duplex. In general, a partial-duplex adapter oligonucleotide comprises one or more single-stranded regions and one or more double-stranded regions. Double-stranded adapter oligonucleotides can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. Often, a single-stranded adapter oligonucleotide comprises two or more sequences that can hybridize with one another. When two such hybridizable sequences are contained in a single-stranded adapter, hybridization yields a hairpin structure (hairpin adapter). When two hybridized regions of an adapter oligonucleotides are separated from one another by a non-hybridized region, a “bubble” structure results. Adapter oligonucleotides comprising a bubble structure consist of a single adapter oligonucleotide comprising internal hybridizations, or comprise two or more adapter oligonucleotides hybridized to one another. Internal sequence hybridization, such as between two hybridizable sequences in adapter oligonucleotides, produce, in some instances, a double-stranded structure in a single-stranded adapter oligonucleotide. Often, adapter oligonucleotides of different kinds are used in combination, such as a hairpin adapter and a double-stranded adapter, or adapters of different sequences. Sometimes, hybridizable sequences in a hairpin adapter include one or both ends of the oligonucleotide. When neither of the ends are included in the hybridizable sequences, both ends are “free” or “overhanging.” When only one end is hybridizable to another sequence in the adapter, the other end forms an overhang, such as a 3′ overhang or a 5′ overhang. When both the 5′-terminal nucleotide and the 3′-terminal nucleotide are included in the hybridizable sequences, such that the 5′-terminal nucleotide and the 3′-terminal nucleotide are complementary and hybridize with one another, the end is referred to as “blunt.” Alternatively, different adapter oligonucleotides are joined to target polynucleotides in sequential reactions or simultaneously. For example, the first and second adapter oligonucleotides is added to the same reaction. Optionally, adapter oligonucleotides are manipulated prior to combining with target polynucleotides. For example, terminal phosphates can be added or removed.

Adapter oligonucleotides contain one or more of a variety of sequence elements, including but not limited to, one or more amplification primer annealing sequences or complements thereof, one or more sequencing primer annealing sequences or complements thereof, one or more barcode sequences, one or more common sequences shared among multiple different adapters or subsets of different adapters, one or more restriction enzyme recognition sites, one or more overhangs complementary to one or more target polynucleotide overhangs, one or more probe binding sites (e.g. for attachment to a sequencing platform, such as a flow cell for massive parallel sequencing, such as developed by Illumina, Inc.), one or more random or near-random sequences (e.g. one or more nucleotides selected at random from a set of two or more different nucleotides at one or more positions, with each of the different nucleotides selected at one or more positions represented in a pool of adapters comprising the random sequence), and combinations thereof. In many examples, two or more sequence elements can be non-adjacent to one another (e.g. separated by one or more nucleotides), adjacent to one another, partially overlapping, or completely overlapping. For example, an amplification primer annealing sequence also serves as a sequencing primer annealing sequence. Sequence elements are located at or near the 3′ end, at or near the 5′ end, or in the interior of the adapter oligonucleotide. When an adapter oligonucleotides can form secondary structure, such as a hairpin, sequence elements can be located partially or completely outside the secondary structure, partially or completely inside the secondary structure, or in between sequences participating in the secondary structure. For example, when an adapter oligonucleotides comprises a hairpin structure, sequence elements can be located partially or completely inside or outside the hybridizable sequences (the “stem”), including in the sequence between the hybridizable sequences (the “loop”). Often, the first adapter oligonucleotides in a plurality of first adapter oligonucleotides having different barcode sequences comprise a sequence element common among all first adapter oligonucleotides in the plurality. Optionally, all second adapter oligonucleotides comprise a sequence element common to all second adapter oligonucleotides that is different from the common sequence element shared by the first adapter oligonucleotides. A difference in sequence elements can be any such that at least a portion of different adapters do not completely align, for example, due to changes in sequence length, deletion or insertion of one or more nucleotides, or a change in the nucleotide composition at one or more nucleotide positions (such as a base change or base modification). Sometimes, an adapter oligonucleotides comprises a 5′ overhang, a 3′ overhang, or both that is complementary to one or more target polynucleotides. Complementary overhangs can be one or more nucleotides in length, including but not limited to 1, 2, 3, 4, 5, 6, 7, 8, 9, 10, 11, 12, 13, 14, 15, or more nucleotides in length. For example, the complementary overhang can be about 1, 2, 3, 4, 5 or 6 nucleotides in length. Complementary overhangs may comprise a fixed sequence. Complementary overhangs may additionally or alternatively comprise a random sequence of one or more nucleotides, such that one or more nucleotides are selected at random from a set of two or more different nucleotides at one or more positions, with each of the different nucleotides selected at one or more positions represented in a pool of adapter oligonucleotides with complementary overhangs comprising the random sequence. Often, an adapter oligonucleotides overhang is complementary to a target polynucleotide overhang produced by restriction endonuclease digestion. Optionally, an adapter oligonucleotide overhang consists of an adenine or a thymine.

Adapter oligonucleotides can have any suitable length, at least sufficient to accommodate the one or more sequence elements of which they are comprised. In some embodiments, adapter oligonucleotides are about, less than about, or more than about 4, 5, 6, 7, 8, 9, 10, 15, 20, 25, 30, 35, 40, 45, 50, 55, 60, 65, 70, 75, 80, 90, 100, 200, or more nucleotides in length. For example, the adapter oligonucleotides are 5 to 15 nucleotides in length. In further examples, the adapter oligonucleotides are about 20 to about 40 nucleotides in length.

Preferably, adapter oligonucleotides are modified, for example by 5′ phosphate excision (via calf alkaline phosphatase treatment, or de novo by synthesis in the absence of such moieties), so that they do not ligate with one another to form multimers. 3′ OH (hydroxyl) moieties are able to ligate to 5′ phosphates on the cleaved nucleic acids, thereby supporting ligation to a first or a second nucleic acid segment.

### Determining Phase Information of a Nucleic Acid Sample

To determine phase information of a nucleic acid sample, a nucleic acid is first acquired, for example by extraction methods discussed herein. In many cases, the nucleic acid is then attached to a solid surface so as to preserve phase information subsequent to cleavage of the nucleic acid molecule. Preferably, the nucleic acid molecule is assembled in vitro with nucleic acid-binding proteins to generate reconstituted chromatin, though other suitable solid surfaces include nucleic acid-binding protein aggregates, nanoparticles, nucleic acid-binding beads, or beads coated using a nucleic acid-binding substance, polymers, synthetic nucleic acid-binding molecules, or other solid or substantially solid affinity molecules. A nucleic acid sample can also be obtained already attached to a solid surface, such as in the case of native chromatin. Native chromatin can be obtained having already been fixed, such as in the form of a formalin-fixed paraffin-embedded (FFPE) or similarly preserved sample.

Following attachment to a nucleic acid binding moiety, the bound nucleic acid molecule can be cleaved. Cleavage is performed with any suitable nucleic acid cleavage entity, including any number of enzymatic and non-enzymatic approaches. Preferably, DNA cleavage is performed with a restriction endonuclease, fragmentase, or transposase. Alternatively or additionally, nucleic acid cleavage is achieved with other restriction enzymes, topoisomerase, non-specific endonuclease, nucleic acid repair enzyme, RNA-guided nuclease, or alternate enzyme. Physical means can also be used to generate cleavage, including mechanical means (e.g., sonication, shear), thermal means (e.g., temperature change), or electromagnetic means (e.g., irradiation, such as UV irradiation). Nucleic acid cleavage produces free nucleic acid ends, either having ‘sticky’ overhangs or blunt ends, depending on the cleavage method used. When sticky overhang ends are generated, the sticky ends are optionally partially filled in to prevent re-ligation. Alternatively, the overhangs are completely filled in to produce blunt ends.

In many cases, overhang ends are partially or completely filled in with dNTPs, which are optionally labeled. In such cases, dNTPs can be biotinylated, sulphated, attached to a fluorophore, dephosphorylated, or any other number of nucleotide modifications. Nucleotide modifications can also include epigenetic modifications, such as methylation (e.g., 5-mC, 5-hmC, 5-fC, 5-caC, 4-mC, 6-mA, 8-oxoG, 8-oxoA). Labels or modifications can be selected from those detectable during sequencing, such as epigenetic modifications detectable by nanopore sequencing; in this way, the locations of ligation junctions can be detected during sequencing. These labels or modifications can also be targeted for binding or enrichment; for example, antibodies targeting methyl-cytosine can be used to capture, target, bind, or label blunt ends filled in with methyl-cytosine. Non-natural nucleotides, non-canonical or modified nucleotides, and nucleic acid analogs can also be used to label the locations of blunt-end fill-in. Non-canonical or modified nucleotides can include pseudouridine (Ψ), dihydrouridine (D), inosine (I), 7-methylguanosine (m7G), xanthine, hypoxanthine, purine, 2,6-diaminopurine, and 6,8-diaminopurine. Nucleic acid analogs can include peptide nucleic acid (PNA), Morpholino and locked nucleic acid (LNA), glycol nucleic acid (GNA), and threose nucleic acid (TNA). In some cases, overhangs are filled in with un-labeled dNTPs, such as dNTPs without biotin. Sometimes, such as cleavage with a transposon, blunt ends are generated that do not require filling in. These free blunt ends are generated when the transposase inserts two unlinked punctuation oligonucleotides. The punctuation oligonucleotides, however, are synthesized to have sticky or blunt ends as desired. Proteins associated with sample nucleic acids, such as histones, can also be modified. For example, histones can be acetylated (e.g., at lysine residues) and/or methylated (e.g., at lysine and arginine residues).

Next, while the cleaved nucleic acid molecule is still bound to the solid surface, the free nucleic acid ends are linked together. Linking occurs, in some cases, through ligation, either between free ends, or with a separate entity, such as an oligonucleotide. In some cases, the oligonucleotide is a punctuation oligonucleotide. In such cases, the punctuation molecule ends are compatible with the free ends of the cleaved nucleic acid molecule. In many cases, the punctuation molecule is dephosphorylated to prevent concatemerization of the oligonucleotides. In most cases, the punctuation molecule is ligated on each end to a free nucleic acid end of the cleaved nucleic acid molecule. In many cases, this ligation step results in rearrangements of the cleaved nucleic acid molecule such that two free ends that were not originally adjacent to one another in the starting nucleic acid molecule are now linked in a paired end.

Following linking of the free ends of the cleaved nucleic acid molecule, the rearranged nucleic acid sample is released from the nucleic acid binding moiety using any number of standard enzymatic and non-enzymatic approaches. For example, in the case of in vitro reconstituted chromatin, the rearranged nucleic acid molecule is released by denaturing or degradation of the nucleic acid-binding proteins. In other examples, cross-linking is reversed. In yet other examples, affinity interactions are reversed or blocked. The released nucleic acid molecule is rearranged compared to the input nucleic acid molecule. In cases where punctuation molecules are used, the resulting rearranged molecule is referred to as a punctuated molecule due to the punctuation oligonucleotides that are interspersed throughout the rearranged nucleic acid molecule. In these cases, the nucleic acid segments flanking the punctuations make up a paired end

During the cleavage and linking steps of the methods disclosed herein, phase information is maintained since the nucleic acid molecule is bound to a solid surface throughout these processes. This can enable the analysis of phase information without relying on information from other markers, such as single nucleotide polymorphisms (SNPs). Using the methods and compositions disclosed herein, in some cases, two nucleic acid segments within the nucleic acid molecule are rearranged such that they are closer in proximity than they were on the original nucleic acid molecule. In many examples, the original separation distance of the two nucleic acid segments in the starting nucleic acid sample is greater than the average read length of standard sequencing technologies. For example, the starting separation distance between the two nucleic acid segments within the input nucleic acid sample is about 10 kb, 12.5 kb, 15 kb, 17.5 kb, 20 kb, 25 kb, 30 kb, 35 kb, 40 kb, 45 kb, 50 kb, 60 kb, 70 kb, 80 kb, 90 kb, 100 kb, 125 kb, 150 kb, 200 kb, 300 kb, 400 kb, 500 kb, 600 kb, 700 kb, 800 kb, 900 kb, 1 Mb, or greater. In preferred examples, the separation distance between the two rearranged DNA segments is less than the average read length of standard sequencing technologies. For example, the distance separating the two rearranged DNA segments within the rearranged DNA molecule is less than about 50 kb, 40 kb, 30 kb, 25 kb, 20 kb, 17 kb, 15 kb, 14 kb, 13 kb, 12 kb, 11 kb, 10 kb, 9 kb, 8 kb, 7 kb, 6 kb, 5 kb, or less. In preferred cases, the separation distance is less than that of the average read length of a long-read sequencing machine. In these cases, when the rearranged DNA sample is released from the nucleic acid binding moiety and sequenced, phase information is determined and sequence information is generated sufficient to generate a de novo sequence scaffold.

### Barcoding a Rearranged Nucleic Acid Molecule

A released rearranged nucleic acid molecule described herein is optionally further processed prior to sequencing. For example, the nucleic acid segments comprised within the rearranged nucleic acid molecule can be barcoded. Barcoding can allow for easier grouping of sequence reads. For example, barcodes can be used to identify sequences originating from the same rearranged nucleic acid molecule. Barcodes can also be used to uniquely identify individual junctions. For example, each junction can be marked with a unique (e.g., randomly generated) barcode which can uniquely identify the junction. Multiple barcodes can be used together, such as a first barcode to identify sequences originating from the same rearranged nucleic acid molecule and a second barcode that uniquely identifies individual junctions.

Barcoding can be achieved through a number of techniques. In some cases, barcodes can be included as a sequence within a punctuation oligo. In other cases, the released rearranged nucleic acid molecule can be contacted to oligonucleotides comprising at least two segments: one segment contains a barcode and a second segment contains a sequence complementary to a punctuation sequence. After annealing to the punctuation sequences, the barcoded oligonucleotides are extended with polymerase to yield barcoded molecules from the same punctuated nucleic acid molecule. Since the punctuated nucleic acid molecule is a rearranged version of the input nucleic acid molecule, in which phase information is preserved, the generated barcoded molecules are also from the same input nucleic acid molecule. These barcoded molecules comprise a barcode sequence, the punctuation complementary sequence, and genomic sequence.

For rearranged nucleic acid molecules with or without punctuation, molecules can be barcoded by other means. For example, rearranged nucleic acid molecules can be contacted with barcoded oligonucleotides which can be extended to incorporate sequence from the rearranged nucleic acid molecule. Barcodes can hybridize to punctuation sequences, to restriction enzyme recognition sites, to sites of interest (e.g., genomic regions of interest), or to random sites (e.g., through a random n-mer sequence on the barcode oligonucleotide). Rearranged nucleic acid molecules can be contacted to the barcodes using appropriate concentrations and/or separations (e.g., spatial or temporal separation) from other rearranged nucleic acid molecules in the sample such that multiple rearranged nucleic acid molecules are not given then same barcode sequence. For example, a solution comprising rearranged nucleic acid molecules can be diluted to such a concentration that only one rearranged nucleic acid molecule will be contacted to a barcode or group of barcodes with a given barcode sequence. Barcodes can be contacted to rearranged nucleic acid molecules in free solution, in fluidic partitions (e.g., droplets or wells), or on an array (e.g., at particular array spots).

Barcoded nucleic acid molecules (e.g., extension products) can be sequenced, for example, on a short-read sequencing machine and phase information is determined by grouping sequence reads having the same barcode into a common phase. Alternatively, prior to sequencing, the barcoded products can be linked together, for example though bulk ligation, to generate long molecules which are sequenced, for example, using long-read sequencing technology. In these cases, the embedded read pairs are identifiable via the amplification adapters and punctuation sequences. Further phase information is obtained from the barcode sequence of the read pair.

Samples from separate cleavage reactions or experiments are sometimes barcoded so as to distinguish data resulting from different experimental conditions. For example, if two or more restriction enzymes or isoschizomers are used in parallel cleavage reactions, then the ligated and/or recovered samples from each individual reaction can be barcoded. In such cases, downstream barcoded libraries can be compared to determine which sequence reads, contigs, and/or scaffolds derive from which experimental conditions. In some cases, the originating strain, species, or sample can be identified based on comparing the presence or absence of sequence reads, contigs, and/or scaffolds from different cleavage reactions using two or more isoschizomers that have differing sensitivity to a base modification, such as methylation.

Barcodes are in some cases added directly to cleaved exposed ends of a digestion reaction, such that all or at least some exposed ends of a complex are commonly barcoded, allowing sequence adjacent to such a barcode to be confidently assigned to a common molecular source.

Determining Phase Information with Paired Ends

Further provided herein are methods and compositions for determining phase information from paired ends. Paired ends can be generated by any of the methods disclosed or those further illustrated in the provided Examples. For example, in the case of a nucleic acid molecule bound to a solid surface which was subsequently cleaved, following re-ligation of free ends, re-ligated nucleic acid segments are released from the solid-phase attached nucleic acid molecule, for example, by restriction digestion. This release results in a plurality of paired ends. In some cases, the paired ends are ligated to amplification adapters, amplified, and sequenced with short reach technology. In these cases, paired ends from multiple different nucleic acid binding moiety-bound nucleic acid molecules are within the sequenced sample. However, it is confidently concluded that for either side of a paired end junction, the junction adjacent sequence is derived from a common phase of a common molecule. In cases where paired ends are linked with a punctuation oligonucleotide, the paired end junction in the sequencing read is identified by the punctuation oligonucleotide sequence. In other cases, the pair ends were linked by modified nucleotides, which can be identified based on the sequence of the modified nucleotides used.

Alternatively, following release of paired ends, the free paired ends can be ligated to amplification adapters and amplified. In these cases, the plurality of paired ends is then bulk ligated together to generate long molecules which are read using long-read sequencing technology. In other examples, released paired ends are bulk ligated to each other without the intervening amplification step. In either case, the embedded read pairs are identifiable via the native DNA sequence adjacent to the linking sequence, such as a punctuation sequence or modified nucleotides. The concatenated paired ends are read on a long-sequence device, and sequence information for multiple junctions is obtained. Since the paired ends derived from multiple different nucleic acid binding moiety-bound DNA molecules, sequences spanning two individual paired ends, such as those flanking amplification adapter sequences, are found to map to multiple different DNA molecules. However, it is confidently concluded that for either side of a paired end junction, the junction-adjacent sequence is derived from a common phase of a common molecule. For example, in the case of paired ends derived from a punctuated molecule, sequences flanking the punctuation sequence are confidently assigned to a common DNA molecule. In preferred cases, because the individual paired ends are concatenated using the methods and compositions disclosed herein, one can sequence multiple paired ends in a single read.

In some examples contigs are clustered by several features. Such features can include presence of specific base modifications, such as methylation, k-mer content, GC content, sequence coverage in the shotgun data, or other features. Clustering can be by any unsupervised clustering algorithm such as k-means clustering, hierarchical clustering, etc. to fractionate contigs into groups that represent species or strains. These groups can then be assembled individually or analyzed unassembled to determine their gene components, biochemical activity, or other characteristics.

### Sequencing

Suitable sequencing methods described herein or otherwise known in the art can be used to obtain sequence information from nucleic acid molecules. Sequencing can be accomplished through classic Sanger sequencing methods. Sequencing can also be accomplished using high-throughput next-generation sequencing systems. Non-limiting examples of next-generation sequencing methods include single-molecule real-time sequencing, ion semiconductor sequencing, pyrosequencing, sequencing by synthesis, sequencing by ligation, and chain termination.

In various embodiments, suitable sequencing methods described herein or otherwise known in the art are used to obtain sequence information from nucleic acid molecules within a sample. Sequencing can be accomplished through classic Sanger sequencing methods which are well known in the art. Sequence can also be accomplished using high-throughput systems some of which allow detection of a sequenced nucleotide immediately after or upon its incorporation into a growing strand, such as detection of sequence in real time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; where the sequencing reads can be at least about 50, about 60, about 70, about 80, about 90, about 100, about 120, about 150, about 180, about 210, about 240, about 270, about 300, about 350, about 400, about 450, about 500, about 600, about 700, about 800, about 900, or about 1000 bases per read.

High-throughput sequencing sometimes involves the use of technology available by Illumina's Genome Analyzer IIX, MiSeq personal sequencer, or HiSeq systems, such as those using HiSeq 2500, HiSeq 1500, HiSeq 2000, or HiSeq 1000 machines. These machines use reversible terminator-based sequencing by synthesis chemistry. These machine can do 200 billion DNA reads or more in eight days. Smaller systems may be utilized for runs within 3, 2, 1 days or less time.

Alternatively, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. Alternatively, an IONPGM™ Sequencer is used. The Ion Torrent Personal Genome Machine (PGM). The PGM can do 10 million reads in two hours.

High-throughput sequencing sometimes involves the use of technology available by Helicos BioSciences Corporation (Cambridge, Massachusetts) such as the Single Molecule Sequencing by Synthesis (SMSS) method. SMSS is unique because it allows for sequencing the entire human genome in up to 24 hours. Finally, SMSS is described in part in US Publication application Nos. 20060024711; 20060024678; 20060012793; 20060012784; and 20050100932.

Alternatively, high-throughput sequencing involves the use of technology available by 454 Lifesciences, Inc. (Branford, Connecticut) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959; and well as in US Publication application Nos. 20020012930; 20030068629; 20030100102; 20030148344; 20040248161; 20050079510, 20050124022; and 20060078909.

High-throughput sequencing is often performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication application Nos. 20040106110; 20030064398; 20030022207; and Constans, A., The Scientist 2003, 17 (13): 36.

The next generation sequencing technique sometimes comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

The next generation sequencing is, in some cases, nanopore sequencing (See, e.g., Soni GV and Meller A. (2007) Clin Chem 53:1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO2). The nanopore can be a hybrid pore (e.g., an integration of a protein pore into a solid-state membrane). The nanopore can be a nanopore with integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. Often, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” Alternatively, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing sometimes comprises DNA nanoball sequencing (as performed, e.g., by Complete Genomics; see e.g., Drmanac et al. (2010) Science 327:78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flow cell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

High-throughput sequencing sometimes takes place using AnyDot.chips (Genovoxx, Germany). In particular, the AnyDot.chips allow for 10×-50× enhancement of nucleotide fluorescence signal detection. AnyDot.chips and methods for using them are described in part in International Publication Application Nos. WO 02088382, WO 03020968, WO 03031947, WO 2005044836, PCT/EP 05/05657, PCT/EP 05/05655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al. Science 24 Mar. 2000; and M. J. Levene, et al. Science 299:682-686, January 2003; as well as US Publication No. 20030044781 and 2006/0078937. Overall such systems involve sequencing a target nucleic acid molecule having a plurality of bases by the temporal addition of bases via a polymerization reaction that is measured on a molecule of nucleic acid, such as the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishable type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

The methods and compositions disclosed herein can be used to generate long DNA molecules comprising rearranged segments compared to the input DNA sample. These molecules are sequences using any number of sequencing technologies. Preferably, the long molecules are sequenced using standard long-read sequencing technologies. Additionally or alternatively, the generated long molecules can be modified as disclosed herein to make them compatible with short-read sequencing technologies.

Exemplary long-read sequencing technologies include but are not limited to nanopore sequencing technologies and other long-read sequencing technologies such as Pacific Biosciences Single Molecule Real Time (SMRT) sequencing. Nanopore sequencing technologies include but are not limited to Oxford Nanopore sequencing technologies (e.g., GridION, MinION) and Genia sequencing technologies.

Sequence read lengths can be at least about 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, or 10 Mb. Sequence read lengths can be about 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, or 10 Mb. In some cases, sequence read lengths are at least about 5 kb. Sometimes, sequence read lengths are about 5 kb.

In some examples, a long rearranged DNA molecule generated using the methods and compositions disclosed herein, is ligated on one end to a sequencing adapter. In preferred examples, the sequencing adapter is a hairpin adapter, resulting in a self-annealing single-stranded molecule harboring an inverted repeat. In these cases, the molecule is fed through a sequencing enzyme and full length sequence of each side of the inverted repeat is obtained. In most cases, the resulting sequence read corresponds to 2×coverage of the DNA molecule, such as a punctuated DNA molecule harboring multiple rearranged segments, each conveying phase information. In favored instances, sufficient sequence is generated to independently generate a de novo scaffold of the nucleic acid sample.

Alternatively, a long rearranged DNA molecule generated using the methods and compositions disclosed herein, is cleaved to form a population of double stranded molecules of a desired length. In these cases, these molecules are ligated on each end to single stranded adapters. The result is a double stranded DNA template capped by hairpin loops at both ends. The circular molecules are sequenced by continuous sequencing technology. Continuous long read sequencing of molecules containing a long double stranded segment results in a single contiguous read of each molecule. Continuous sequencing of molecules containing a short double stranded segment results in multiple reads of the molecule, which are used either alone or along with continuous long read sequence information to confirm a consensus sequence of the molecule. In most cases, genomic segment borders marked by punctuation oligonucleotides are identified, and it is concluded that sequence adjacent to a punctuation border is in phase. In preferred cases, sufficient sequence is generated to independently generate a de novo scaffold of the nucleic acid sample.

Rearranged nucleic acid molecules are often selected for sequencing based on length. Length-based selection can be used to select for rearranged nucleic acid molecules that contain more rearranged segments, so that shorter rearranged nucleic acid molecules containing only a few rearranged segments are not sequenced or are sequenced in fewer numbers. Rearranged nucleic acid molecules containing more rearranged segments can provide more phasing information than those molecules containing fewer rearranged segments. Rearranged nucleic acid molecules can be selected for those that contain at least 1, 2, 3, 4, 5, 6, 7, 8, 9, 10, or more rearranged segments. For example, rearranged nucleic acid molecules can be selected for a length of at least 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, or more. Length-based selection can be a firm exclusion, excluding 100% of rearranged nucleic acid molecules below the chosen length. Alternatively, length-based selection can be an enrichment for longer molecules, removing at least 99.999%, 99.99%, 99.9%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 5%, 4%, 3%, 2%, or 1% of rearranged nucleic acid molecules below the chosen length. Length selection of nucleic acids can be performed by a variety of techniques, including but not limited to electrophoresis (e.g., gel or capillary), filtration, bead binding (e.g., SPRI bead size selection), and flow-based methods.

### Microbes

The microbes detected herein are contemplated to include bacteria, viruses, fungi, mold, or any other microscopic organism or a combination thereof.

Microbes detected in biomedical samples herein, such as for example a biological fluid or a solid sample including but not limited to saliva, blood, stool, plant material or soil, often is at least one bacterial or other microbial species associated with a medical or agronomic condition. Non-limiting examples of clinically relevant microorganisms include Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaninogenicus (now known as Prevotella melaninogenica), Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae (previously called Chlamydia pneumoniae), Chlamydophila psittaci (previously called Chlamydia psittaci), Clostridium botulinum, Clostridium difficile, Clostridium perfringens (previously called Clostridium welchii), Clostridium tetani, Corynebacterium diphtheriae, Corynebacterium fusiforme, Coxiella burnetii, Ehrlichia chaffeensis, Enterobacter cloacae, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Neisseria gonorrhoeae, Neisseria meningitidis, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsii, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Vibrio cholerae, Vibrio comma, Vibrio parahaemolyticus, Vibrio vulnificus, Wolbachia, Yersinia enterocolitica, Yersinia pestis, and Yersinia pseudotuberculosis.

Sometimes, a microbe detected in a biomedical sample, such as for example a biological fluid or a solid sample including but not limited to saliva, blood, and stool, is at least virus associated with a medical condition. In some aspects, viruses are DNA viruses. Alternatively, viruses are RNA viruses. Human viral infections can have a zoonotic, or wild or domestic animal, origin. Several zoonotic viruses are transmitted to humans directly via contact with an animal or indirectly via exposure to the urine or feces of infected animals or the bite of a bloodsucking arthropod. If a virus is able to adapt and replicate in its new human host, human-to-human transmissions may occur. Often, a microbe detected in a biomedical sample is a virus having a zoonotic origin.

A microbe detected in a biomedical sample, such as for example a biological fluid or a solid sample including but not limited to saliva, blood, and stool, sometimes is at least fungus associated with a medical condition. Non-limiting examples of clinically relevant fungal genuses include Aspergillus, Basidiobolus, Blastomyces, Candida, Chrysosporium, Coccidioides, Conidiobolus, Cryptococcus, Epidermophyton, Histoplasma, Microsporum, Pneumocystis, Sporothrix, and Trichophyton.

A microbe detected in a food sample, such a food sample suspected of causing illness, sometimes is a pathogenic bacterium, virus, or parasite. Non-limiting examples of pathogenic bacteria, viruses, or parasites that can cause illness include Salmonella species such as S. enterica and S. bongori; Campylobacter species such as C. jejuni, C. coli, and C. fetus; Yersinia species such as Y. enterocolitica and Y. pseudotuberculosis; Shigella species such as S. sonnei, S. boydii, S. flexneri, and S. dysenteriae; Vibrio species such as V. parahaemolyticus, Vibrio cholerae Serogroups 01 and 0139, Vibrio cholerae Serogroups non-01 and non-0139, Vibrio vulnificus; Coxiella species such as C. burnetii; Mycobacterium species such as M. bovis which is the causative agent of tuberculosis in cattle but can also infect humans; Brucella species such as B. melitensis, B. abortus, B. suis, B. neotomae, B. canis, and B. ovis; Cronobacter species (formery Enterobacter sakazakii); Aeromonas species such as A. hydrophila; Plesiomonas species such as P. shigelloides; Francisella species such as F. tularensis; Clostridium species such as C. perfringens and C. botulinum; Staphylococcus species such as S. aureus; Bacillus species such as B. cereus; Listeria species such as L. monocytogenes; Streptococcus species such as S. pyogenes of Group A; Noroviruses (NoV, groups GI, GII, GIII, GIV, and GV); Hepatitis A virus (HAV, genotypes I-VI); Hepatitis E virus (HEV); Reoviridae viruses such as Rotavirus; Astroviridae viruses such as Astroviruses; Calciviridae viruses such as Sapoviruses; Adenoviridae viruses such as Enteric adenoviruses; Parvoviridae viruses such as Parvoviruses; and Picornaviridae viruses such as Aichi virus.

A benefit of the methods disclosed herein is that they facilitate the detection of a microbe or pathogen of unknown identity in a sample, and the assembly of the sequence information for that unknown microbe or pathogen into a partially or fully assembled genome, alone or in combination with additional sequence information such as concurrently generated sequence information generated by shotgun sequencing or other means. Accordingly, approaches disclosed herein are not limited to the detection of one or more of the organisms listed immediately above; on the contrary, through the methods disclosed herein, one is able to identify and determine substantial partial or total genome information for an unknown pathogen in the list above, or an organism not on the list above, or an organism for which no sequence information is available, or an organism that is not known to science.

The methods disclosed herein are applicable to a number of heterogeneous nucleic acid samples, such as exploratory surveys of gut microflora; pathogen detection in a sick individual or population, such as a population suffering from an epidemic of unknown cause; the assay of a heterogeneous nucleic acid sample for the presence of nucleic acids having linkage information characteristic of a known individual; or the detection of the microbe or microbes responsible for antibiotic resistance in an individual exhibiting an antibiotic resistant infection. A common aspect of many of these embodiments is that they benefit from the generation of long-range linkage information such as that suitable for the assembly of shotgun sequence information into contigs, scaffolds or partial or complete genome sequences. Shotgun or other high-throughput sequence information is relevant to at least some of the issues listed above, but substantial benefit is gained from the result of the practice of the methods disclosed herein, to assemble shotgun sequence into larger phased nucleic acid assemblies, up to and including partial, substantially complete or complete genomes. Accordingly, use of the methods disclosed herein provides substantially more than the practice of shotgun sequencing alone on the heterogeneous samples as known in the art.

In addition to illness caused by direct bacterial infection after ingesting contaminated and/or spoiled food, microbes can produce toxins, such as an enterotoxin, that cause illness. In some aspects, a microbe detected in a food sample can produce a toxin such as an enterotoxin, which is a protein exotoxin that targets the intestines, and mycotoxin, which is a toxic secondary metabolite produced by organisms of the fungi kingdom, commonly known as molds.

A benefit of the present disclosure is that it enables one to obtain long-range genome contiguity information for a heterogeneous sample without relying upon previously or even concurrently generated sequence information for the genome or genomes to be assembled. Scaffolds, representing genomes or chromosomes of organisms in the sample, are assembled using commonly tagged reads, such as reads sharing a common oligo tag or paired-end reads that are ligated or otherwise fused to one another, thereby indicating that commonly tagged sequence information arises from a common genomic or chromosomal molecule.

Accordingly, scaffold information is generated without reliance upon previously generated contig or other sequence read information. There are a number of benefits of de novo scaffold information. For example, sequence reads can be assigned to common scaffolds even if no previous sequence information is available, such that entirely new genomes are scaffolded without reliance upon previous sequencing efforts. This benefit is particularly useful when a heterogeneous sample comprises an unknown, uncultured or unculturable organism. Whereas a sequencing project relying upon untargeted sequence read generation may generate a collection of sequence reads that are not assigned to any known contig sequence, there would be little or no information relating to the number or identity of the unknown organisms from which the sequence reads were obtained. They could, for example, represent a single individual, a population of individuals of a common species having a high degree of heterogeneity or heterozygosity in genomic sequence, a complex of closely related species, or a complex of different species. Relying solely on sequence read information, one would not easily distinguish among the aforementioned scenarios.

However, using the methods or compositions as disclosed herein, one is able to distinguish among, for example, a sample comprising clonal duplicates of a common genotype or genome, from a sample comprising a heterogeneous population of representatives of a single species, from a sample comprising loosely related organisms of different species, or combinations of these scenarios. Relying upon sequence similarity to assemble contigs rather than independently generating scaffold information, one is challenged to distinguish heterozygosity from sequencing error. Even assuming that no substantial sequencing error occurs, one is challenged to even estimate the number of genotypes from which closely-related genome information is obtained. One cannot, for example, distinguish a sample comprising two widely divergent representatives of a single species, heterozygous relative to one another at a number of distinct loci, from a sample comprising a broad diversity of closely related genotypes, each differing from the others at one or only a few loci. Using sequence read information alone, both of these scenarios appear as a single contig assembly having substantial allelic diversity. However, using the methods and compositions disclosed herein, one is able to determine with confidence which alleles map to a common scaffold, even if the alleles are separated by considerable regions of uniform or unknown sequence.

This benefit of the data generated herein is particularly useful in some cases when a heterogeneous sample comprises a viral population, such as a DNA-genome based viral population or a retrovirus or other RNA-based viral population is studied (via reverse transcription of the RNA genomes or, alternately or in combination, assembling complexes on RNA in the sample). As viral populations are often considerably heterogeneous, understanding the distribution of the heterogeneity within the population (either among a few highly divergent populations or among a large number of closely related populations) is of particular benefit in selecting a treatment target and in tracing the origin of the virus in the heterogeneous sample being studied.

This is not to say that the compositions and methods disclosed herein are incompatible with contig information or concurrently generated sequence reads. On the contrary, the scaffolding information generated through use of the methods and compositions herein are particularly suited for improved contig assembly or contig arrangement into scaffolds. Indeed, concurrently generated sequence read information is assembled into contigs in some embodiments of the disclosure herein. Sequence read information is generated in parallel, using traditional sequencing approaches such as next-generation sequencing approaches. Alternately or in combination, paired read or oligo-tagged read information is used as sequence information itself to generate contigs ‘traditionally’ using aligned overlapping sequence. This information is further used to position contigs relative to one another in light of the scaffolding information generated through the compositions and methods disclosed herein.

The disclosure herein is further clarified in reference to a partial list of numbered embodiments as follows. 1. A method of genome assembly comprising: a) obtaining a plurality of contigs; b) complexing naked DNA from a sample with isolated nuclear proteins to form reconstituted chromatin; c) generating a plurality of read pairs from data produced by probing the physical layout of the reconstituted chromatin, wherein generating said plurality of read pairs comprises applying at least two restriction enzymes to said reconstituted chromatin, and wherein at least one of said restriction enzymes is modification-sensitive; d) mapping the plurality of read pairs to the plurality of contigs thereby producing read-mapping data; and e) arranging the contigs using the read-mapping data to assemble the contigs into a genome assembly, such that contigs having common read pairs are positioned to determine a path through the contigs that represents their order to the genome. 2. The method of embodiment 1, wherein said plurality of contigs is generated by using a shotgun sequencing method, comprising: a) fragmenting a subject's DNA into random fragments of indeterminate size; b) sequencing the fragments using high throughput sequence methods to generate a plurality of sequencing reads; and c) assembling the sequencing reads so as to form the plurality of contigs. 3. The method of embodiment 1 or embodiment 2, wherein generating a plurality of read pairs from data produced by probing the physical layout of reconstituted chromatin comprises using crosslinking. 4. The method of any one of embodiments 1 to 3, wherein at least two of said restriction enzymes are isoschizomers. 5. The method of any one of embodiments 1 to 4, wherein at least one of said restriction enzymes is not an isoschizomer of at least one other of said restriction enzymes. 6. The method of any one of embodiments 1 to 5, wherein at least two of said restriction enzymes recognize a particular sequence. 7. The method of embodiment 6, wherein the particular sequence is a GATC sequence. 8. The method of any one of embodiments 1 to 7, wherein at least two of said restriction enzymes are BfuCI enzymes. 9. The method of any one of embodiments 1 to 8, wherein at least two of said restriction enzymes are selected from a group consisting of: MboI, DpnI, Sau3AI, and BfuCI. 10. The method of any one of embodiments 1 to 9, wherein at least one of said isoschizomers is modification-sensitive. 11. The method of any one of embodiments 1 to 10, wherein at least two of said isoschizomers are modification-sensitive. 12. The method of any one of embodiments 1 to 11, wherein at least three of said restriction enzymes are modification-sensitive. 13. The method of embodiment 11 or embodiment 12, wherein at least one of said modification-sensitive restriction enzyme has activity in the presence of base modification. 14. The method of embodiment 13, wherein said base modification is necessary for activity. 15. The method of any one of embodiments 11 to 14, wherein said base modification precludes activity. 16. The method of any one of embodiments 11 to 15, wherein said base modification is a methylation of a nucleoside. 17. The method of any one of embodiments 11 to 16, wherein said base modification is selected from a group consisting of: CpG methylation of cytosine, methylation of adenosine, and methylation of cytosine. 18. The method of any one of embodiments 1 to 17, wherein generating a plurality of read pairs from data produced by probing the physical layout of reconstituted chromatin comprises: a) crosslinking reconstituted chromatin with a fixative agent to form DNA-protein cross links; b) cutting the cross-linked DNA-Protein with one or more restriction enzymes so as to generate a plurality of DNA-Protein complexes comprising sticky ends; c) cutting the cross-linked DNA-Protein with one or more of the condition-sensitive enzymes so as to generate a plurality of DNA-Protein complexes comprising sticky ends; d) filling in the sticky ends with nucleotides containing one or more markers to create blunt ends that are then ligated together; e) fragmenting the plurality of DNA-protein complexes into fragments; f) pulling down junction-containing fragments by using the one or more markers; and g) sequencing the junction containing fragments using high throughput sequencing methods to generate the plurality of read pairs. 19. The method of embodiment 18, wherein said one or more markers is a biotinylated nucleotide. 20. The method of any one of embodiments 1 to 19, wherein the isolated nuclear proteins comprise isolated histones. 21. The method of any one of embodiments 1 to 20, wherein for the plurality of read pairs, read pairs are weighted by taking a function of a read's distance to the edge of a mapped contig so as to incorporate a higher probability of shorter contacts than longer contacts. 22. The method of any one of embodiments 1 to 21, wherein the method provides for the genome assembly of a human subject, and wherein the plurality of read pairs is generated by using the human subject's reconstituted chromatin made from the subject's naked DNA. 23. The method of any one of embodiments 1 to 22, wherein the method further comprises: a) identifying one or more sites of heterozygosity in the plurality of read pairs; and b) identifying read pairs that comprise a pair of heterozygous sites, wherein phasing data for allelic variants can be determined from the identification of the pair of heterozygous sites. 24. The method of any one of embodiments 1 to 23, wherein said arranging the contigs using the read pair data comprises: a) constructing an adjacency matrix of contigs using the readmapping data; and b) analyzing the adjacency matrix to determine a path through the contigs that represents their order in the genome. 25. The method of embodiment 24, comprising analyzing the adjacency matrix to determine a path through the contigs that represents their order and orientation to the genome. 26. The method of embodiment 24 or embodiment 25, wherein a read pair is weighted as a function of the distance from the mapped position of its first read on a first contig to the edge of that first contig and the distance from the mapped position of its second read on a second contig to the edge of that second contig. 27. The method of any one of embodiments 1 to 26, wherein the plurality of contigs is generated from the human subject's DNA. 28. The method of any one of embodiments 1 to 27, wherein the genome assembly represents the contigs' order and orientation. 29. The method of any one of embodiments 1 to 28, wherein a read pair is weighted as a function of the distance from the mapped position of its first read on a first contig to the edge of that first contig and the distance from the mapped position of its second read on a second contig to the edge of that second contig. 30. The method of any one of embodiments 1 to 29, wherein read pairs that map to different contigs provide data about which contigs are adjacent in a correct genome assembly. 31. The method of any one of embodiments 1 to 30, wherein said sample is taken from a complex biological environment. 32. The method of embodiment 31, wherein the method provides for the genome assembly of genomes in said sample taken from a complex biological environment, and wherein the plurality of read pairs is generated from reconstituted chromatin made from the sample's naked DNA. 33. The method of embodiment 31 or embodiment 32, wherein the complex biological environment comprises human gut microbes. 34. The method of any one of embodiments 31 to 33, wherein the complex biological environment comprises human skin microbes. 35. The method of any one of embodiments 31 to 34, wherein the complex biological environment comprises waste site microbes. 36. The method of any one of embodiments 31 to 35, wherein the complex biological environment comprises an ecological environment. 37. The method of any one of embodiments 1 to 36, wherein the plurality of contigs is generated from the sample's DNA. 38. The method of any one of embodiments 1 to 37, wherein the genome assemblies represent the contigs' order and orientation. 39. A method of categorizing a contig as arising from a nucleic acid having a particular base modification, comprising: a) obtaining a first population of read pair sequence information generated by contacting a nucleic acid sample aliquot using a modification-sensitive endonuclease; b) obtaining a second population of read pair sequence information generated by contacting a nucleic acid sample aliquot using a modification-insensitive endonuclease, wherein the modification-sensitive endonuclease and the condition-insensitive endonuclease are isoschizomers; c) identifying a contig to which first population read pairs and second population read pairs both map; and d) categorizing the contig as arising from a nucleic acid having the particular base modification because first population read pairs and second population read pairs mapping to the contig do not share common read pair junctions at a frequency observed for first population read pair junctions in the first population of read pair sequence information. 40. The method of embodiment 39, comprising assigning the contig to a scaffold comprising contigs having the particular base modification. 41. The method of embodiment 39 or embodiment 40, comprising assigning the contig to a genome comprising contigs having the particular base modification. 42. The method of any one of embodiments 39 to 41, comprising assigning the contig to a genome of an organism for which the particular base modification is relatively abundant. 43. The method of any one of embodiments 39 to 42, wherein the particular base modification is selected from the list consisting of methylation, hydroxymethylation, and oxidation. 44. The method of embodiment 43, wherein the particular base modification is methylation. 45. The method of any one of embodiments 39 to 44, wherein first population read pairs and second population read pairs mapping to the contig do not share common read pair junctions. 46. The method of any one of embodiments 39 to 45, wherein first population read pairs and second population read pairs mapping to the contig share common read pair junctions at a rate that is lower than the frequency of common read pair junctions in the first population of read pair sequence information. 47. The method of any one of embodiments 39 to 46, wherein first population read pairs and second population read pairs mapping to the contig share common read pair junctions at a rate that is lower than the frequency of common read pair junctions in the second population of read pair sequence information. 48. The method of any one of embodiments 39 to 47, wherein the nucleic acid sample aliquot using a modification-sensitive endonuclease and the nucleic acid sample aliquot using a modification-insensitive endonuclease are taken from a sample taken from a complex biological environment. 49. The method of any one of embodiments 39 to 48, wherein the method provides for the genome assembly of genomes in said sample taken from a complex biological environment, and wherein the plurality of read pairs is generated from reconstituted chromatin made from the sample's naked DNA. 50. The method of embodiment 48 or embodiment 49, wherein the complex biological environment comprises human gut microbes. 51. The method of any one of embodiments 48 to 50, wherein the complex biological environment comprises human skin microbes. 52. The method of any one of embodiments 48 to 51, wherein the complex biological environment comprises waste site microbes. 53. The method of any one of embodiments 48 to 52, wherein the complex biological environment comprises an ecological environment. 54. The method of any one of embodiments 39 to 53, wherein the plurality of contigs is generated from the sample's DNA. 55. The method of any one of embodiments 39 to 54, wherein the genome assemblies represent the contigs' order and orientation. 56. The method of any one of embodiments 39 to 55, the method further comprising: a) digesting a sample using a modification-sensitive enzyme; b) tagging cleavage products; c) pulling down said tagged products; d) sequencing at least a recognizable part of the tagged products; and e) assigning contigs to which the tagged products map to a common source. 57. A method of grouping contigs comprising: a) identifying a feature common to a subset of contigs in a contig population; and b) assigning the subset of contigs to a common group. 58. The method of embodiment 57, wherein the feature comprises methylation status. 59. The method of embodiment 57 or embodiment 58, wherein the feature comprises GC content. 60. The method of any one of embodiments 57 to 59, wherein the feature comprises k-mer content. 61. The method of any one of embodiments 57 to 60, wherein the feature comprises sequence coverage in a shotgun sequence dataset. 62. The method of any one of embodiments 57 to 61, wherein identifying the feature comprises: a) obtaining a first population of read pair sequence information generated by contacting a nucleic acid sample aliquot using a modification-sensitive endonuclease; b) obtaining a second population of read pair sequence information generated by contacting a nucleic acid sample aliquot using a modification-insensitive endonuclease, wherein the modification-sensitive endonuclease and the modification-insensitive endonuclease are isoschizomers; c) identifying a contig to which first population read pairs and second population read pairs both map; and d) categorizing the contig as arising from a nucleic acid having the modification because first population read pairs and second population read pairs mapping to the contig do not share common read pair junctions at a frequency observed for first population read pair junctions in the first population of read pair sequence information. 63. The method of any one of embodiments 57 to 62, wherein the common group comprises a scaffold. 64. The method of any one of embodiments 57 to 63, wherein the common group comprises a chromosome. 65. The method of embodiment 64, wherein the chromosome is differentially methylated in a genome. 66. The method of embodiment 64 or embodiment 65, wherein the chromosome is a sex chromosome. 67. The method of any one of embodiments 64 to 66, wherein the chromosome is a y-chromosome. 68. The method of any one of embodiments 64 to 67, wherein the chromosome is an x-chromosome. 69. The method of any one of embodiments 57 to 68, wherein the common group comprises a genome. 70. The method of embodiment 69, wherein the genome is differentially methylated. 71. The method of any one of embodiments 57 to 70, wherein the nucleic acid sample aliquot using a modification-sensitive endonuclease and the nucleic acid sample aliquot using a modification-insensitive endonuclease are taken from a sample taken from a complex biological environment. 72. The method of any one of embodiments 57 to 71, wherein the method provides for the genome assembly of genomes in said sample taken from a complex biological environment, and wherein the plurality of read pairs is generated from reconstituted chromatin made from the sample's naked DNA. 73. The method of embodiment 71 or embodiment 72, wherein the complex biological environment comprises human gut microbes. 74. The method of any one of embodiments 71 to 73, wherein the complex biological environment comprises human skin microbes. 75. The method of any one of embodiments 71 to 74, wherein the complex biological environment comprises waste site microbes. 76. The method of any one of embodiments 71 to 75, wherein the complex biological environment comprises an ecological environment. 77. The method of any one of embodiments 57 to 76, wherein the plurality of contigs is generated from the sample's DNA. 78. The method of any one of embodiments 57 to 77, wherein the genome assemblies represent the contigs' order and orientation. 79. The method of any one of embodiments 57 to 78, the method further comprising: a) digesting a sample using a modification-sensitive enzyme; b) tagging cleavage products, pulling down tagged products; c) sequencing at least a recognizable part of the tagged products; and d) assigning contigs to which the tagged products map to a common source. 80. A method of determining genomic linkage information for a heterogeneous nucleic acid sample comprising: a) obtaining a stabilized heterogeneous nucleic acid sample; b) contacting the stabilized sample to cleave double-stranded DNA in the stabilized sample, wherein contacting said stabilized sample comprises applying at least two restriction enzymes to said stabilized sample, and wherein at least one of said restriction enzymes is modification-sensitive; c) tagging exposed DNA ends; d) ligating tagged exposed DNA ends to form tagged paired ends; e) obtaining a first sequence and a second sequence from a first side and a second side of said ligated paired ends to generate a plurality of paired sequence reads; f) assigning each half of a paired sequence read of the plurality of sequence reads to a common nucleic acid molecule of origin. 81. The method of embodiment 80, wherein the heterogeneous nucleic acid sample is obtained from blood, sweat, urine or stool. 82. The method of embodiment 80 or embodiment 81, wherein the stabilized sample has been cross-linked. 83. The method of any one of embodiments 80 to 82, wherein the stabilized sample has been contacted to formaldehyde. 84. The method of any one of embodiments 80 to 83, wherein the stabilized sample has been contacted to psoralen. 85. The method of any one of embodiments 80 to 84, wherein the stabilized sample has been exposed to UV radiation. 86. The method of any one of embodiments 80 to 85, wherein the sample has been contacted to a DNA binding moiety. 87. The method of embodiment 86, wherein the DNA binding moiety comprises a histone. 88. The method of any one of embodiments 80 to 87, wherein at least two of said restriction enzymes are isoschizomers. 89. The method of any one of embodiments 80 to 88, wherein at least one of said restriction enzymes is not an isoschizomer of at least one other of said restriction enzymes. 90. The method of any one of embodiments 80 to 89, wherein at least two of said restriction enzymes recognize a particular sequence. 91. The method of embodiment 90, wherein the particular sequence is a GATC sequence. 92. The method of any one of embodiments 80 to 91, wherein at least two of said restriction enzymes are BfuCI enzymes. 93. The method of any one of embodiments 80 to 92, wherein at least two of said restriction enzymes are selected from a group consisting of: MboI, DpnI, Sau3AI, and BfuCI. 94. The method of any one of embodiments 80 to 93, wherein at least one of said isoschizomers is modification-sensitive. 95. The method of any one of embodiments 80 to 94, wherein at least two of said isoschizomers are modification-sensitive. 96. The method of any one of embodiments 80 to 95, wherein at least three of said restriction enzymes are modification-sensitive. 97. The method of any one of embodiments 80 to 96, wherein at least one of said modification-sensitive restriction enzyme has activity in the presence of base modification. 98. The method of embodiment 97, wherein said base modification is necessary for activity. 99. The method of embodiment 97, wherein said base modification precludes activity. 100. The method of any one of embodiments 97 to 99, wherein said base modification is a methylation of a nucleoside. 101. The method of any one of embodiments 97 to 100, wherein said base modification is selected from a group consisting of: CpG methylation of cytosine, methylation of adenosine, and methylation of cytosine. 102. The method of any one of embodiments 80 to 101, wherein tagging exposed DNA ends comprises adding a biotin moiety to an exposed DNA end. 103. The method of any one of embodiments 80 to 102, wherein searching the paired sequence against a DNA database. 104. The method of any one of embodiments 80 to 103, wherein the common nucleic acid molecule of origin maps to a single individual. 105. The method of any one of embodiments 80 to 104, wherein the common nucleic acid molecule of origin identifies a subset of a population. 106. A method of determining genomic linkage information for a heterogeneous nucleic acid sample comprising: a) obtaining a stabilized heterogeneous nucleic acid sample; b) treating the stabilized sample to cleave double-stranded DNA in the stabilized sample, wherein contacting said stabilized sample comprises applying at least two restriction enzymes to said stabilized sample, and wherein at least one of said restriction enzymes is modification-sensitive; c) tagging exposed DNA ends of a first portion of the stabilized sample using a first barcode tag and tagging exposed ends of a second portion of the stabilized sample using a second barcode tag; d) sequencing across barcode tagged ends to generate a plurality of barcode tagged sequence reads; e) assigning commonly tagged sequence reads to a common nucleic acid molecule of origin. 107. The method of embodiment 106, wherein the heterogeneous nucleic acid sample is obtained from blood, sweat, urine or stool. 108. The method of embodiment 106 or embodiment 107, wherein the stabilized sample has been cross-linked. 109. The method of any one of embodiments 106 to 108, wherein the stabilized sample has been contacted to formaldehyde. 110. The method of any one of embodiments 106 to 109, wherein the stabilized sample has been contacted to psoralen. 111. The method of any one of embodiments 106 to 110, wherein the stabilized sample has been exposed to UV radiation. 112. The method of any one of embodiments 106 to 111, wherein the sample has been contacted to a DNA binding moiety. 113. The method of embodiment 112, wherein the DNA binding moiety comprises a histone. 114. The method of any one of embodiments 106 to 113, wherein at least two of said restriction enzymes are isoschizomers. 115. The method of any one of embodiments 106 to 114, wherein at least one of said restriction enzymes is not an isoschizomer of at least one other of said restriction enzymes. 116. The method of any one of embodiments 106 to 115, wherein at least two of said restriction enzymes recognize a particular sequence. 117. The method of embodiment 116, wherein the particular sequence is a GATC sequence. 118. The method of any one of embodiments 106 to 117, wherein at least two of said restriction enzymes are BfuCI enzymes. 119. The method of any one of embodiments 106 to 118, wherein at least two of said restriction enzymes are selected from a group consisting of: MboI, DpnI, Sau3AI, and BfuCI. 120. The method of any one of embodiments 106 to 119, wherein at least one of said isoschizomers is modification-sensitive. 121. The method of any one of embodiments 106 to 120, wherein at least two of said isoschizomers are modification-sensitive. 122. The method of any one of embodiments 106 to 121, wherein at least three of said restriction enzymes are modification-sensitive. 123. The method of any one of embodiments 106 to 122, wherein at least one of said modification-sensitive restriction enzyme has activity in the presence of base modification. 124. The method of embodiment 123, wherein said base modification is necessary for activity. 125. The method of embodiment 123 or claim 124, wherein said base modification precludes activity. 126. The method of any one of embodiments 123 to 125, wherein said base modification is a methylation of a nucleoside. 127. The method of any one of embodiments 106 to 126, wherein said base modification is selected from a group consisting of: CpG methylation of cytosine, methylation of adenosine, and methylation of cytosine. 128. The method of any one of embodiments 106 to 127, wherein tagging exposed DNA ends comprises adding a biotin moiety to an exposed DNA end. 129. The method of any one of embodiments 106 to 128, wherein searching the paired sequence against a DNA database. 130. The method of any one of embodiments 106 to 129, wherein the common nucleic acid molecule of origin maps to a single individual. 131. The method of any one of embodiments 106 to 130, wherein the common nucleic acid molecule of origin identifies a subset of a population. 132. A method of determining genomic linkage information for a heterogeneous nucleic acid sample comprising: a) stabilizing the heterogeneous nucleic acid sample; b) treating the stabilized sample to cleave double-stranded DNA in the stabilized sample, thereby generating exposed DNA ends, wherein contacting said stabilized sample comprises applying at least two restriction enzymes to said stabilized sample, and wherein at least one of said restriction enzymes is modification-sensitive; c) tagging at least a portion of the exposed DNA ends; d) ligating the tagged exposed DNA ends to form tagged paired ends; e) obtaining a first sequence and a second sequence from a first side and a second side of said ligated paired ends to generate a plurality of read-pairs; f) assigning each half of a read-pair to a common nucleic acid molecule of origin. 133. The method of embodiment 132, wherein the heterogeneous nucleic acid sample is obtained from blood, sweat, urine or stool. 134. The method of embodiment 132 or embodiment 133, wherein the stabilized sample has been cross-linked. 135. The method of any one of embodiments 132 to 134, wherein the stabilized sample has been contacted to formaldehyde. 136. The method of any one of embodiments 132 to 135, wherein the stabilized sample has been contacted to psoralen. 137. The method of any one of embodiments 132 to 136, wherein the stabilized sample has been exposed to UV radiation. 138. The method of any one of embodiments 132 to 137, wherein the sample has been contacted to a DNA binding moiety. 139. The method of embodiment 138, wherein the DNA binding moiety comprises a histone. 140. The method of any one of embodiments 132 to 139, wherein at least two of said restriction enzymes are isoschizomers. 141. The method of any one of embodiments 132 to 140, wherein at least one of said restriction enzymes is not an isoschizomer of at least one other of said restriction enzymes. 142. The method of any one of embodiments 132 to 141, wherein at least two of said restriction enzymes recognize a particular sequence. 143. The method of embodiment 142, wherein the particular sequence is a GATC sequence. 144. The method of any one of embodiments 132 to 143, wherein at least two of said restriction enzymes are BfuCI enzymes. 145. The method of any one of embodiments 132 to 144, wherein at least two of said restriction enzymes are selected from a group consisting of: MboI, DpnI, Sau3 AI, and BfuCI. 146. The method of any one of embodiments 132 to 145, wherein at least one of said isoschizomers is modification-sensitive. 147. The method of any one of embodiments 132 to 146, wherein at least two of said isoschizomers are modification-sensitive. 148. The method of any one of embodiments 132 to 147, wherein at least three of said restriction enzymes are modification-sensitive. 149. The method of any one of embodiments 132 to 148, wherein at least one of said modification-sensitive restriction enzyme has activity in the presence of base modification. 150. The method of embodiment 149, wherein said base modification is necessary for activity. 151. The method of embodiment 149, wherein said base modification precludes activity. 152. The method of any one of embodiments 149 to 151, wherein said base modification is a methylation of a nucleoside. 153. The method of any one of embodiments 149 to 152, wherein said base modification is selected from a group consisting of: CpG methylation of cytosine, methylation of adenosine, and methylation of cytosine. 154. The method of any one of embodiments 132 to 153, wherein tagging exposed DNA ends comprises adding a biotin moiety to an exposed DNA end. 155. The method of any one of embodiments 132 to 154, wherein searching the paired sequence against a DNA database. 156. The method of any one of embodiments 132 to 155, wherein the common nucleic acid molecule of origin maps to a single individual. 157. The method of any one of embodiments 132 to 156, wherein the common nucleic acid molecule of origin identifies a subset of a population. 158. A method for meta-genomics assemblies, comprising: a) collecting microbes from an environment; b) obtaining a plurality of contigs from the microbes; c) generating a plurality of read pairs from data produced by probing the physical layout of reconstituted chromatin, wherein generating said plurality of read pairs comprises applying at least two restriction enzymes to said reconstituted chromatin, and wherein at least one of said restriction enzymes is modification-sensitive; d) mapping the plurality of read pairs to the plurality of contigs thereby producing read-mapping data, wherein read pairs mapping to different contigs indicate which contigs are from the same species. 159. The method of embodiment 158, wherein the microbes are collected from a human gut. 160. A method for detecting a bacterial infectious agent, comprising: a) obtaining a plurality of contigs from the bacterial infectious agent; b) generating a plurality of read pairs from data produced by probing the physical layout of reconstituted chromatin, wherein generating said plurality of read pairs comprises applying at least two restriction enzymes to said reconstituted chromatin, and wherein at least one of said restriction enzymes is modification-sensitive; c) mapping the plurality of read pairs to the plurality of contigs thereby producing read-mapping data; d) arranging the contigs using the read-mapping data to assemble the contigs into a genome assembly; and e) using the genome assembly to determine presence of the bacterial infectious agent. 161. A method of obtaining genomic sequence information from an organism comprising: a) obtaining a stabilized sample from said organism; b) contacting the stabilized sample to cleave double-stranded DNA in the stabilized sample, thereby generating exposed DNA ends, wherein contacting said stabilized sample comprises applying at least two restriction enzymes to said stabilized sample, and wherein at least one of said restriction enzymes is modification-sensitive; c) tagging at least a portion of the exposed DNA ends to generate tagged DNA segments; d) sequencing said tagged DNA segments and thereby obtaining tagged sequences; e) mapping said tagged sequences to generate genomic sequence information of said organism, wherein said genomic sequence information covers at least 75% of the genome of said organism. 162. The method of embodiment 161, wherein said organism is collected from a heterogeneous sample. 163. The method of embodiment 162, wherein said heterogeneous sample comprises at least 1000 organisms each comprising a different genome. 164. The method of any one of embodiments 161 to 163, wherein said stabilized sample is obtained by contacting DNA from said organism to a DNA binding moiety. 165. The method of embodiment 164, wherein said DNA binding moiety is a histone. 166. The method of embodiment 164, wherein said DNA binding moiety is a nanoparticle. 167. The method of embodiment 164, wherein said DNA binding moiety is a transposase. 168. The method of any one of embodiments 161 to 167, wherein at least two of said restriction enzymes are isoschizomers. 169. The method of any one of embodiments 161 to 168, wherein at least one of said restriction enzymes is not an isoschizomer of at least one other of said restriction enzymes. 170. The method of any one of embodiments 161 to 169, wherein at least two of said restriction enzymes recognize a particular sequence. 171. The method of embodiment 170, wherein the particular sequence is a GATC sequence. 172. The method of any one of embodiments 161 to 171, wherein at least two of said restriction enzymes are BfuCI enzymes. 173. The method of any one of embodiments 161 to 172, wherein at least two of said restriction enzymes are selected from a group consisting of: MboI, DpnI, Sau3AI, and BfuCI. 174. The method of any one of embodiments 161 to 173, wherein at least one of said isoschizomers is modification-sensitive. 175. The method of any one of embodiments 161 to 174, wherein at least two of said isoschizomers are modification-sensitive. 176. The method of any one of embodiments 161 to 175, wherein at least three of said restriction enzymes are modification-sensitive. 177. The method of any one of embodiments 161 to 176, wherein at least one of said modification-sensitive restriction enzyme has activity in the presence of base modification. 178. The method of embodiment 177, wherein said base modification is necessary for activity. 179. The method of embodiment 177, wherein said base modification precludes activity. 180. The method of any one of embodiments 177 to 179, wherein said base modification is a methylation of a nucleoside. 181. The method of any one of embodiments 177 to 180, wherein said base modification is selected from a group consisting of: CpG methylation of cytosine, methylation of adenosine, and methylation of cytosine. 182. The method of any one of embodiments 161 to 181, wherein said exposed DNA ends are tagged using a transposase. 183. The method of any one of embodiments 161 to 182, wherein said portion of exposed DNA ends are tagged by linking said exposed DNA ends to another exposed DNA end. 184. The method of any one of embodiments 161 to 183, wherein said portion of exposed DNA ends are linked to said other exposed DNA ends using a ligase. 185. The method of any one of embodiments 161 to 184, wherein said genomic sequence information is generated without using additional contig sequences obtained from said genome. 186. A method of generating long-distance phase information from a first DNA molecule, comprising: a) providing a first DNA molecule having a first segment and a second segment, wherein the first segment and the second segment are not adjacent on the first DNA molecule; b) contacting the first DNA molecule to a DNA binding moiety such that the first segment and the second segment are bound to the DNA binding moiety independent of a common phosphodiester backbone of the first DNA molecule; c) cleaving the first DNA molecule such that the first segment and the second segment are not joined by a common phosphodiester backbone, wherein cleaving the first DNA molecule comprises applying at least two restriction enzymes to said stabilized sample, and wherein at least one of said restriction enzymes is modification-sensitive; d) attaching the first segment to the second segment via a phosphodiester bond to form a reassembled first DNA molecule; and e) sequencing at least 4 kb of consecutive sequence of the reassembled first DNA molecule comprising a junction between the first segment and the second segment in a single sequencing read, wherein first segment sequence and second segment sequence represent long-distance phase information from a first DNA molecule. 187. The method of embodiment 186, wherein the DNA binding moiety comprises a plurality of DNA-binding molecules. 188. The method of embodiment 186 or embodiment 187, wherein contacting the first DNA molecule to a plurality of DNA-binding molecules comprises contacting to a population of DNA-binding proteins. 189. The method of embodiment 188, wherein the population of DNA-binding proteins comprises nuclear proteins. 190. The method of embodiment 188, wherein the population of DNA-binding proteins comprises nucleosomes. 191. The method of embodiment 188, wherein the population of DNA-binding proteins comprises histones. 192. The method of any one of embodiments 186 to 191, wherein contacting the first DNA molecule to a plurality of DNA-binding moieties comprises contacting to a population of DNA-binding nanoparticles. 193. The method of any one of embodiments 186 to 192, wherein the first DNA molecule has a third segment not adjacent on the first DNA molecule to the first segment or the second segment, wherein the contacting in (b) is conducted such that the third segment is bound to the DNA binding moiety independent of the common phosphodiester backbone of the first DNA molecule, wherein the cleaving in (c) is conducted such that the third segment is not joined by a common phosphodiester backbone to the first segment and the second segment, wherein the attaching comprises attaching the third segment to the second segment via a phosphodiester bond to form the reassembled first DNA molecule, and wherein the consecutive sequence sequenced in (e) comprises a junction between the second segment and the third segment in a single sequencing read. 194. The method of any one of embodiments 186 to 193, comprising contacting the first DNA molecule to a cross-linking agent. 195. The method of any one of embodiments 186 to 194, comprising contacting the first DNA molecule to a cross-linking agent. 196. The method of embodiment 195, wherein the cross-linking agent is formaldehyde. 197. The method of embodiment 195 or embodiment 196, wherein the cross-linking agent is formaldehyde. 198. The method of any one of embodiments 186 to 197, wherein the DNA binding moiety is bound to a surface comprising a plurality of DNA binding moieties. 199. The method of any one of embodiments 186 to 198, wherein the DNA binding moiety is bound to a solid framework comprising a bead. 200. The method of any one of embodiments 186 to 199, wherein at least two of said restriction enzymes are isoschizomers. 201. The method of any one of embodiments 186 to 200, wherein at least one of said restriction enzymes is not an isoschizomer of at least one other of said restriction enzymes. 202. The method of any one of embodiments 186 to 201, wherein at least two of said restriction enzymes recognize a particular sequence. 203. The method of embodiment 202, wherein the particular sequence is a GATC sequence. 204. The method of any one of embodiments 186 to 203, wherein at least two of said restriction enzymes are BfuCI enzymes. 205. The method of any one of embodiments 186 to 204, wherein at least two of said restriction enzymes are selected from a group consisting of: MboI, DpnI, Sau3AI, and BfuCI. 206. The method of any one of embodiments 186 to 205, wherein at least one of said isoschizomers is modification-sensitive. 207. The method of any one of embodiments 186 to 206, wherein at least two of said isoschizomers are modification-sensitive. 208. The method of any one of embodiments 186 to 207, wherein at least three of said restriction enzymes are modification-sensitive. 209. The method of any one of embodiments 186 to 208, wherein at least one of said modification-sensitive restriction enzyme has activity in the presence of base modification. 210. The method of embodiment 209, wherein said base modification is necessary for activity. 211. The method of embodiment 209, wherein said base modification precludes activity. 212. The method of any one of embodiments 209 to 211, wherein said base modification is a methylation of a nucleoside. 213. The method of any one of embodiments 209 to 212, wherein said base modification is selected from a group consisting of: CpG methylation of cytosine, methylation of adenosine, and methylation of cytosine. 214. The method of any one of embodiments 186 to 213, comprising adding a tag to at least one exposed end. 215. The method of embodiment 214, wherein the tag comprises a labeled base. 216. The method of embodiment 214 or embodiment 215, wherein the tag comprises a methylated base. 217. The method of any one of embodiments 214 to 216, wherein the tag comprises a biotinylated base. 218. The method of any one of embodiments 214 to 217, wherein the tag comprises uridine. 219. The method of any one of embodiments 214 to 218, wherein the tag comprises a noncanonical base. 220. The method of any one of embodiments 214 to 219, wherein the tag generates a blunt ended exposed end. 221. The method of any one of embodiments 186 to 220, comprising adding at least one base to a recessed strand of a first segment sticky end. 222. The method of any one of embodiments 186 to 221, comprising adding a linker oligo comprising an overhang that anneals to the first segment sticky end. 223. The method of embodiment 222, wherein the linker oligo comprises an overhang that anneals to the first segment sticky end and an overhang that anneals to the second segment sticky end. 224. The method of embodiment 222 or embodiment 223, wherein the linker oligo does not comprise two 5′ phosphate moieties. 225. The method of any one of embodiments 186 to 224, wherein attaching comprises ligating. 226. The method of any one of embodiments 186 to 225, wherein attaching comprises DNA single strand nick repair. 227. The method of any one of embodiments 186 to 226, wherein the first segment and the second segment are separated by at least 10 kb on the first DNA molecule prior to cleaving the first DNA molecule. 228. The method of any one of embodiments 186 to 227, wherein the first segment and the second segment are separated by at least 15 kb on the first DNA molecule prior to cleaving the first DNA molecule. 229. The method of any one of embodiments 186 to 228, wherein the first segment and the second segment are separated by at least 30 kb on the first DNA molecule prior to cleaving the first DNA molecule. 230. The method of any one of embodiments 186 to 229, wherein the first segment and the second segment are separated by at least 50 kb on the first DNA molecule prior to cleaving the first DNA molecule. 231. The method of any one of embodiments 186 to 230, wherein the first segment and the second segment are separated by at least 100 kb on the first DNA molecule prior to cleaving the first DNA molecule. 232. The method of any one of embodiments 186 to 231, wherein the sequencing comprises single molecule long read sequencing. 233. The method of embodiment 232, wherein the long-read sequencing comprises a read of at least 5 kb. 234. The method of embodiment 232 or embodiment 233, wherein the long-read sequencing comprises a read of at least 10 kb. 235. The method of any one of embodiments 186 to 234, wherein the first reassembled DNA molecule comprises a hairpin moiety linking a 5′ end to a 3′ end at one end of the first DNA molecule. 236. The method of any one of embodiments 186 to 235, comprising sequencing a second reassembled version of the first DNA molecule. 237. The method of any one of embodiments 186 to 236, wherein the first segment and the second segment are each at least 500 bp. 238. The method of any one of embodiments 186 to 237, wherein the first segment, the second segment, and the third segment are each at least 500 bp.

### Examples

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1. Combinatorial Restriction Enzyme Usage

A combination restriction enzyme approach as described herein were used to generate shotgun data. Naked DNA samples were cut separately using a combination of restriction enzymes as shown in Table 1. The restriction products were labeled with biotin. Streptavidin pull-down was used to enrich for DNA fragments that had been cut with each enzyme, whose base-modification specificity is known. Mapping these reads back to contigs revealed the base-modification status of the genome in which it occurs.

Shotgun sequencing libraries were generated using a standard approach and the libraries were sequences and the contigs were assembled.

Chicago libraries were then generated using a combination of isoschizomer enzymes that differ in their sensitivity to base modification. Four Chicago libraries were generated using MboI, DpnII, Sau3AI, and a combination of all three enzymes. Each of these restriction enzymes cuts GATC, but either will not cut this sequence in the presence of specific base modifications or require specific base modifications as shown in Table 2.

TABLE 2
Isoschizomers and their base-modification sensitivities

| | Restriction | dam | dcm | CpG |
|---|---|---|---|---|
| Enzyme | site | methylation | methylation | methylation |
| MboI | GATC | Blocked | Not blocked | Blocked |
| DpnI | GATC | Required | Not blocked | Blocked |
| Sau3AI | GATC | Not blocked | Not blocked | Blocked |

During the proximity ligation protocol, DNA was cut using the indicated restriction enzymes to generate free ends. These free ends were then marked with a biotinylated nucleotide and ligated. After ligation, the biotin mark was used to purify ligation-containing fragments.

Each Chicago library was prepared separately from the same in vitro chromatin preparation. Each Chicago library was individually barcoded, pooled with the others, and then sequenced as a pool or separately.

The sequence data from the resulting Chicago libraries were contrasted to reveal which assembly components (contigs or scaffolds) derive from strains or species that have similar base-modification activities. Samples containing a methylation state that blocks the activity of the restriction enzyme in that reaction were not cleaved and therefore sequences were from that sample were absent or present at a relatively low level in the generated Chicago libraries.

Contigs were clustered according to their methylation state based on the corresponding sequencing reads being present in Chicago libraries generated by the specified restriction enzyme (See FIG. 1A and FIG. 1B).

FIG. 1A and FIG. 1B depict the identification of assembled sequences that derive from strains or species that are dam methylated. FIG. 1A shows a metagenomic assembly, as generated using the protocol in FIG. 2B, and was made using a cocktail of all isoschizomer restriction enzymes listed in Table 2. The ratio of Chicago/shotgun reads, per contig (y-axis) is nearly constant across contigs because all instances of GATC are cut with at least one of the restriction enzymes. FIG. 1B shows that when the Chicago library is generated using an enzyme, MboI for example, that is sensitive to dam methylation, the ratio of Chicago to shotgun reads is severely reduced in genomes that are dam methylated. In this way, those components can be identified as belonging to strains or species that use dam methylation.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of mapping a sequence to a nucleic acid molecule, comprising the steps of obtaining a nucleic acid sample comprising a first nucleic acid molecule comprising a first region and a second region;
contacting said nucleic acid sample with a binding agent such that said first region and said second region of said first nucleic acid molecule are redundantly bound independently of a phosphodiester backbone of said first nucleic acid molecule;
digesting said nucleic acid sample to produce at least one double strand break of known end sequence between said first region and said second region of said first nucleic acid molecule;
contacting said nucleic acid sample to a population of oligonucleotides comprising a first plurality of oligonucleotides,
wherein each of said first plurality of oligonucleotides comprises a) a 3′ annealing region capable of annealing to said double strand break, and b) a first molecular tag sequence 5′ of said annealing region,
ligating said known end sequence to at least one oligonucleotide of said population of oligonucleotides to form a ligation product;
separating said binding agent from said first nucleic acid molecule; and
sequencing said first ligation product.

2. The method of claim 1, wherein a second sequence comprising said first molecular tag corresponds to a sequence of said first nucleic acid molecule.

3. The method of claim 1, wherein said nucleic acid sample is subjected to fragmentation prior to contacting with said binding agent.

4. The method of claim 1, wherein said population of oligonucleotides comprises a second plurality of oligonucleotides, wherein each of said second plurality of oligonucleotides comprises
a) a 3′ annealing region capable of annealing to said double strand break, and
b) a second molecular tag sequence 5′ of said annealing region, having a sequence different from that of said first molecular tag.

5. The method of claim 4, wherein said second plurality of oligonucleotides is spatially separate from said first plurality of oligonucleotides.

6. The method of claim 1, wherein the population of oligonucleotides is attached to a solid surface.

7. The method of claim 6, wherein said solid surface is a nucleic acid array.

8. A method for generating labeled polynucleotides from a first DNA molecule, wherein said first DNA molecule comprises a first sequence segment and a second sequence segment, said method comprising:
a. crosslinking said first sequence segment and said second sequence segment outside of a cell;
b. adding said first sequence segment and said second sequence segment to a first resolved locus comprising a plurality of binding probes; and
c. generating a first labeled polynucleotide comprising a first label and a first complement sequence, and a second labeled polynucleotide comprising a second label and a second complement sequence.

9. The method of claim 8, further comprising obtaining sequence information of said first labeled polynucleotide and said second labeled polynucleotide.

10. The method of claim 9, further comprising using said sequence information to associate said first sequence segment and said second sequence segment.

11. The method of claim 8, wherein said first sequence segment and said second sequence segment is cross-linked to a plurality of association molecules.

12. The method of claim 11, wherein said association molecules comprise peptides or proteins.

13. The method of claim 8, wherein said first resolved locus is located on a substrate.

14. The method of claim 13, wherein said substrate is a microarray.

15. The method of claim 14, wherein said microarray comprises one or more elements selected from the group consisting of a linker, a primer, a barcode and a capture sequence.

16. A kit comprising:
a) a crosslinking agent;
b) a nucleic acid molecule; and
c) an endonuclease.
